(19) United States
(12) Patent Application Publication
(10) Pub. No.: US 2022/0047742 A1
(43) Pub. Date: Feb. 17, 2022

(54) METHOD AND APPARATUS FOR PROVIDING SELF-STERILIZING SANITARY BARRIER

(71) Applicant: UNIVERSITY OF CENTRAL FLORIDA RESEARCH FOUNDATION, INC., Orlando, FL (US)

(72) Inventors: Hwan CHOI, Orlando, FL (US); Zhishan GUO, Orlando, FL (US); Sepehr RAMEZANI, Orlando, FL (US); Matthew CRAWFORD, Orlando, FL (US)

(21) Appl. No.: 17/403,426

(22) Filed: Aug. 16, 2021

Related U.S. Application Data
(60) Provisional application No. 63/065,628, filed on Aug. 14, 2020.
(60) Provisional application No. 63/163,141, filed on Mar. 19, 2021.

(51) Int. Cl. A61L 2/10

(52) U.S. Cl. CPC A61L 2/10

(57) ABSTRACT

A method and apparatus for providing a self-sterilizing sanitary barrier is disclosed. The apparatus includes a housing defining an interior space configured to receive a mask sheet such that the mask sheet is movable from a first position covering a portion of a face of a user to a second position within the interior space of the housing. The apparatus also includes a light source mounted within the interior space and configured to emit radiation to sterilize the mask sheet in the second position. The method includes steps including donning the apparatus on the user and moving the mask from the first position to the second position. The method further includes emitting radiation from the light source within the housing to sterilize the mask sheet in the second position.

### CROSS-REFERENCE TO RELATED APPLICATIONS

This application is a Non-Provisional Patent Application that claims priority under 35 USC § 119 to U.S. Provisional Application No. 63/065,628 filed on 14 Aug. 2020 and U.S. Provisional Application No. 63/163,141 filed Mar. 19, 2021 and are incorporated herein by reference in their entirety.

### BACKGROUND

Respiratory infectious diseases such as corona virus disease 2019 (COVID-19), severe acute respiratory syndrome (SARS), and middle east respiratory syndrome (MERS) are highly contagious and can be lethal to human beings. The World Health Organization (WHO) and Centers for Disease Control and Prevention (CDC) strongly recommend wearing a mask to prevent infections and mitigate the spread of these diseases.

### SUMMARY

The inventors of the present invention recognized that current designs for conventional face masks typically include a single sheet and two ear loops to cover the mouth, nose and ears. FIGS. 1A and 1B depict scenarios where conventional face masks are typically employed, e.g., to protect the user from pathogens (e.g. airborne viral particles) in the surroundings of the user (FIG. 1A) and/or to protect the surroundings (e.g. other people) of the user from pathogens in the user (FIG. 1B). While these conventional masks are easy to don and doff, these mask designs have limited effectiveness at preventing disease for various reasons. One reason for this limited effectiveness is that users often forget to wear the mask when appropriate. For example, people in work environments usually take off their mask when performing isolated tasks (e.g., having meals or operating a vehicle). Frequently, people forget to reapply their masks because they temporarily set the mask down (e.g., on a desk or in the car). Another reason for the limited effectiveness of conventional masks is that users unintentionally contaminate their masks. For example, some people grab the sheet of their mask with their hands, as they don and doff the mask, leading to contamination of the mask. Also, workers or people with frequent field activates sometimes wear a mask for long periods of time during their operating hours without changing or sterilizing their mask. These poor mask handling techniques can reduce the mask's effectiveness in disease prevention. Thus, it is crucial to improve mask design to help keep the masks on and sterilized during working activities.

In one embodiment, an apparatus for providing a self-sterilizing sanitary barrier is presented. The apparatus includes a housing defining an interior space configured to receive a mask sheet such that the mask sheet is movable from a first position covering a portion of a face of a user to a second position within the interior space of the housing. The apparatus also includes a light source mounted within the interior space and configured to emit radiation to sterilize the mask sheet in the second position.

In another embodiment, a method for providing a self-sterilizing sanitary barrier is presented. The method involves steps including donning the apparatus on the user and moving the mask from the first position to the second position. The method further includes emitting radiation from the light source within the housing to sterilize the mask sheet in the second position.

Still other aspects, features, and advantages are readily apparent from the following detailed description, simply by illustrating a number of particular embodiments and implementations, including the best mode contemplated for carrying out the invention. Other embodiments are also capable of other and different features and advantages, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

Embodiments are illustrated by way of example, and not by way of limitation, in the figures of the accompanying drawings in which like reference numerals refer to similar elements and in which:

FIG. 1A is an image that illustrates an example of a donned mask sheet on a user to protect the user from pathogens in the surroundings;

FIG. 1B is an image that illustrates an example of a donned mask sheet on a user to protect the surroundings from pathogens in the user;

FIG. 2A is an image that illustrates an example of a perspective view of a mask sheet of the apparatus in a first position covering a portion of a face of the user and a second position within a housing of the apparatus, according to an embodiment;

FIG. 2B is an image that illustrates an example of the mask sheet of the apparatus wrapped around an inner rod that is rotatably coupled within the housing, according to an embodiment;

FIG. 2C is an image that illustrates an example of a light source of the apparatus emitting light at the mask sheet within the housing to sterilize the mask sheet, according to an embodiment;

FIG. 2D is a block diagram that illustrates an example of the apparatus of FIGS. 2A and 2B, according to an embodiment;

FIG. 2E is an image that illustrates an example of a side perspective view of the mask sheet, the inner rod and the housing of the apparatus, according to an embodiment;

FIGS. 2F through 2H are images that illustrate an example of accessories to be used with the apparatus of FIGS. 2A and 2B, according to an embodiment;

FIG. 3A is an image that illustrates an example of an overlap or folded area in the mask sheet around the inner rod of the apparatus of FIG. 2A;

FIGS. 3B through 3H are images that illustrate an example of various views of an apparatus for providing a self-sterilizing sanitary barrier, according to an embodiment;

FIGS. 3I through 3J are images that illustrate an example of various views of an apparatus for providing a self-sterilizing sanitary barrier, according to an embodiment;

FIG. 3K is a block diagram that illustrates an example of the apparatus of FIGS. 3B through 3H and the apparatus of FIGS. 3I through 3J, according to an embodiment;

FIGS. 3L through 3M are images that illustrate an example of a rolling mechanism of the apparatus of FIGS. 3B through 3H and FIGS. 3I through 3J, according to an embodiment;

FIGS. 3N through 3Q are images that illustrate an example of the mask sheet of the apparatus of FIGS. 3I through 3J being moved between the first and second positions, according to an embodiment;

FIGS. 3R and 3S are images that illustrate an example of a gradient figure of UV energy dosage of the respective back and front of the mask sheet, according to an embodiment;

FIG. 4A is an image that illustrates an example of an apparatus for providing a self-sterilizing sanitary barrier, according to an embodiment;

FIG. 4B is an image that illustrates an example of the mask sheet of the apparatus of FIG. 4A in the extended position, according to an embodiment;

FIG. 4C is an image that illustrate an example of an exploded view of the apparatus of FIG. 4A, according to an embodiment;

FIG. 4D is an image that illustrates an example of a perspective view of the apparatus of FIGS. 4A through 4C, according to an embodiment;

FIG. 5 is a graph that illustrates an example of a best fit curve between conductance and light intensity at sensors within the apparatus of FIG. 4A, according to an embodiment;

FIGS. 6A and 6B are images that illustrate an example of a gradient figure of UV energy dosage of the respective back and front of the mask sheet, according to an embodiment;

FIGS. 7A and 7B are images that illustrate an example of perspective views of the respective front and back side of the mask sheet within the apparatus of FIG. 4A, according to an embodiment;

FIG. 7C is an image that illustrates an example of a spatial arrangement of sensors positioned on the front and back side of the mask sheet in FIGS. 7A and 7B, according to an embodiment;

FIG. 7D is an image that illustrates an example of a perspective view of components used to calibrate the apparatus of FIG. 4A, according to an embodiment;

FIG. 8A is a flow chart that illustrates an example method for using the mounting the apparatus to the system of FIG. 1A and using the system, according to an embodiment;

FIG. 8B is a flow chart that illustrates an example method for calibrating the apparatus of FIG. 4A, according to an embodiment;

FIG. 9 is a block diagram that illustrates a computer system upon which an embodiment of the invention may be implemented;

FIG. 10 illustrates a chip set upon which an embodiment of the invention may be implemented; and

FIG. 11 is a diagram of components of a mobile terminal (e.g., cell phone handset) for communications, which is capable of operating in the system, according to one embodiment.

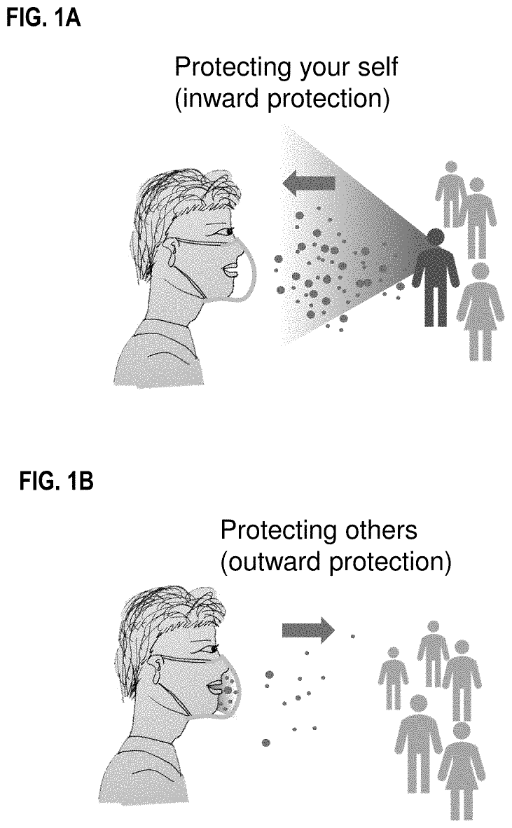

### DETAILED DESCRIPTION

A method and apparatus are described for providing a self-sterilizing sanitary barrier. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus, a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5λ to 2λ, for example, about 100 implies a value in a range from 50 to 200. Moreover, all ranges disclosed herein are to be understood to encompass any and all sub-ranges subsumed therein. For example, a range of “less than 10” for a positive only parameter can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

Some embodiments of the invention are described below in the context of providing a sanitary barrier (e.g., facial mask) to protect the user from pathogens (e.g. viral particles) in the surroundings of the user and/or to protect the surroundings of the user from pathogens (e.g. viral particles) from the user. For purposes of this invention, “pathogen” means any bacterium, virus (e.g., SARS-COV2), or other microorganism or any variant thereof (e.g., Delta variant of SARS-COV2) that can cause disease (e.g. COVID-19). For purposes of this description, “mask sheet” means material that is used to cover a portion of a body (e.g., face) of a user and is effective at reducing and/or minimizing the transmission of pathogens between the body (e.g. face) of the user and the surroundings of the user. In one example embodiment, “mask sheet” is made from one or more of high-thread-count cotton fabric, silk, chiffon, polyester and/or polypropylene fiber. In some embodiments, the “mask sheet” has one or more dimensions (e.g., width, length, etc.) that are sized to cover a portion of a face of a user (e.g. nose and mouth, eyes nose and mouth, etc.). In other embodiments, the embodiments are described below in the context of providing a self-sterilizing sanitary barrier for any portion of a body of the user (e.g., other than the face).

### 1. OVERVIEW

Since being first reported in December 2019, coronavirus disease 2019 (COVID-19) has continued to spread worldwide with more than 164 million confirmed cases and 3.4 million related deaths as of May 20, 2021 [1]. Severe acute respiratory syndrome coronavirus 2 (SARS-CoV-2) is the beta coronavirus which causes COVID-19 and is able to be spread via multiple modes of transmission, including direct contact and through airborne particulates [2]. As a result, the importance of methods to reduce the spread of all types of infectious disease between individuals has been emphasized. While mask wearing as a mitigation technique has been effective, it has also revealed the shortcomings of current mask technologies.

Previous studies have shown masks to be effective at blocking the release of respiratory particles into the wearer's nearby environment, while also acting as a filter that can reduce the exposure to these infectious droplets[3,4]. However, the ability of a mask to protect the wearer is reduced when it is not used properly. One of the most common faults in the use of a protective face covering or mask sheet is contamination. Improper handling and storage of a mask when not in use, as well as prolonged use without sanitization, and physical touching of the mask material with infected hands can all contribute to the contamination of the mask sheet and the exposure of the wearer to these pathogens [5].

Current mask technology is limited to two broad categories of face coverings: disposable and reusable. Both types have unique advantages and disadvantages, allowing for significant room for improvement. Disposable masks are designed for single-use and can easily be thrown away and replaced when contaminated, making them useful in clinical settings. However, the United Nations reported in July 2020 that an estimated 75% of the waste from disposable masks will end up in landfills or in the oceans. [6] A September 2020 study estimated that 16,659 tons of medical waste is produced daily solely in Asia. [7]. With the spread of the COVID-19 pandemic, the rate of production of medical waste in the United States has increased from 5 million tons/year to 30 million tons/year. [8] It is estimated that 75-90% of this waste is composed of nonhazardous paper and plastic materials, which includes disposable face masks and other personal protective equipment (PPE). [9] Furthermore, the World Health Organization expects the demand for disposable PPE to increase by up to 20% by 2025 as additional emphasis is placed on reducing the spread of infectious disease. [10].

Reusable masks significantly reduce the amount of plastic waste produced; however, the repeated use of a single face covering or mask sheet can lead to the accumulation of harmful viruses and bacteria on the protective material. [11] In order to maintain the function of reusable face coverings, they must be constantly washed, resulting in the expenditure of large amounts of water and electricity, while also taking extended periods of time for the sanitization process to be completed, making them expensive and impractical in clinical settings.

When considering alternative methods to sanitize a face covering, few options match effectiveness with practicality. Antiseptic solutions such as bleach are primarily used to disinfect hard surfaces and require copious amounts of water when used on porous materials. [12] Autoclaves are commonly used to sterilize laboratory materials; however, these machines are expensive and impractical for use in many clinical settings and by the public.[13] One method that combines efficiency and functionality is the use of concentrated ultraviolet radiation to sanitize surfaces. [14, 15, 16] Ultraviolet C (UVC) radiation has been directly tested on SARS-CoV-2, and has been shown to hinder viral replication by damaging the nucleic acid genetic material.[15] It was found that UVC with a wavelength of about 254 nm and at an energy dosage of 5 mJ/cm² inactivates 99% of the virus on surfaces.[15]. The effectiveness was also shown of using ultraviolet C radiation at a wavelength of about 222 nm to destroy the outer shell of coronaviruses similar to SARS-CoV-2. [16] It was found that an energy dosage of 2 mJ/cm² successfully inactivated 99.9% of the alpha coronavirus HCoV-229E and 99.99% of the beta coronavirus HCoV-OC43. [16] Germicidal UV light has long been proven useful in disinfecting surfaces to reduce the spread of other pathogens such as Mycobacterium tuberculosis, H1N1 Influenza, and Escherichia coli. [14, 16, 17]

Given the importance of facial coverings in order to reduce the transmission of COVID-19 between individuals, insufficient attention is given to the improvement of current mask design function and sustainability. There is a need for a mask that is more easily sanitized and can still be easily put on and removed, while also promoting healthy mask wearing. As a result, the embodiments of the present invention disclose an improved mask, to further combat the spread of COVID-19 and other infectious diseases and reduce unnecessary waste.

With the spread of COVID-19, significant emphasis has been placed on mitigation techniques such as mask wearing to slow infectious disease transmission. Widespread use of face coverings has revealed challenges such as mask contamination and waste, presenting an opportunity to improve the current technologies. In response, the embodiments of the present invention disclose a novel, reusable mask sheet that can be quickly disinfected using an apparatus including a light source (e.g., UV light source, such as an array of ultraviolet C lamps) contained within a wearable housing. A sensor (e.g., nanomembrane UVC sensor) was used to quantify the intensity of germicidal radiation at a plurality (e.g., 18) different locations on the mask sheet and determine the necessary exposure time to inactivate a pathogen (e.g., SARS-CoV-2) in addition to other viruses and bacteria. After experimentation, it was found that the improved apparatus disclosed herein successfully provided germicidal radiation to all areas of the mask sheet and will inactivate different pathogens in different time periods (e.g., SARS-CoV-2 in approximately 180 seconds, H1N1 Influenza in 130 seconds, and Mycobacterium tuberculosis in 113 seconds), proving that this design is effective at eliminating a variety of pathogens and can serve as an alternative to traditional waste-producing disposable face masks. The accessibility, ease of use, and speed of sanitization supports the wide application of the apparatus in both clinical and public settings.

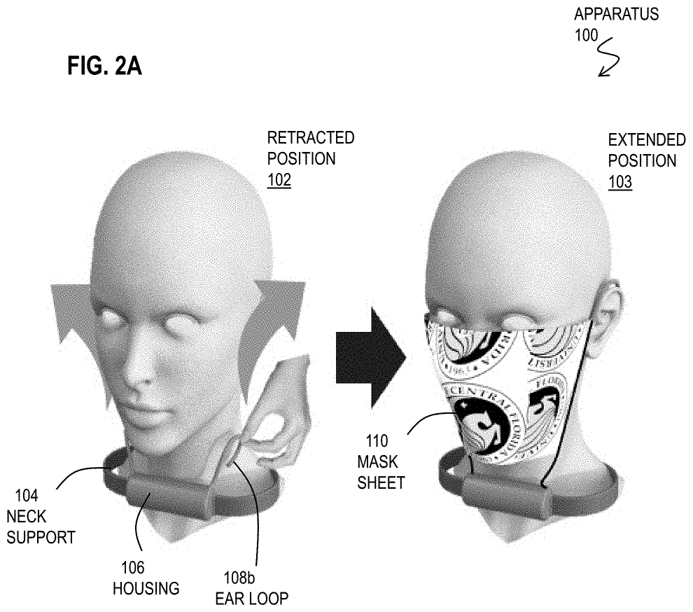

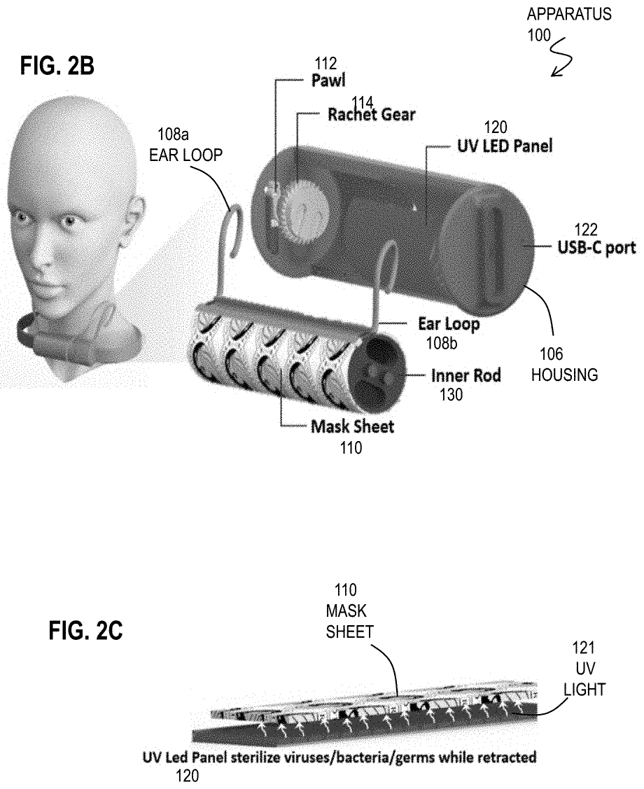

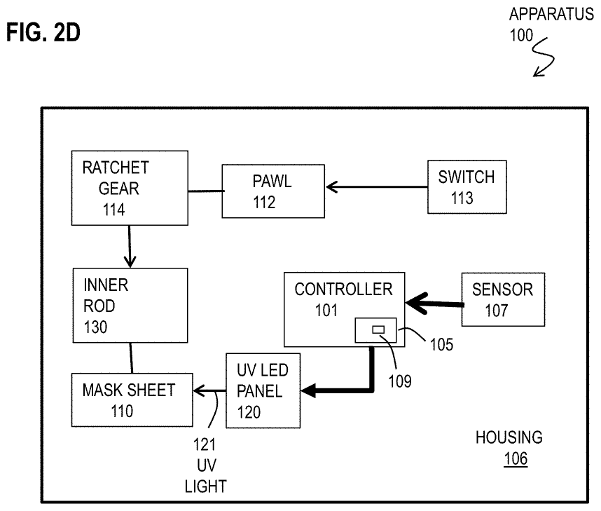

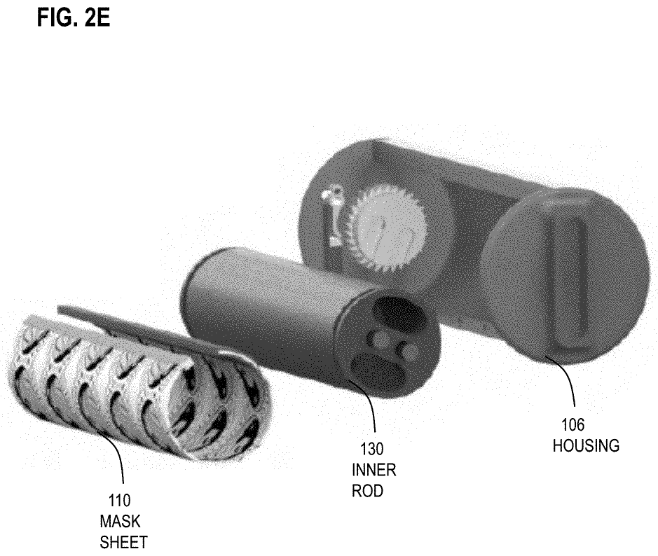

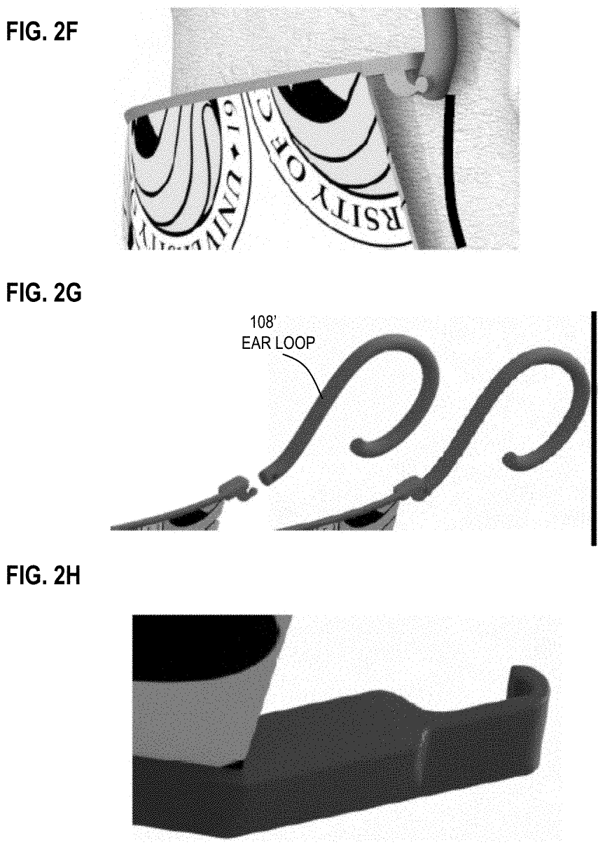

FIG. 2A is an image that illustrates an example of a perspective view of an apparatus 100 for providing a self-sterilizing sanitary barrier, according to an embodiment. In an embodiment, the apparatus 100 includes a neck support 104 (e.g., a necklace to encircle the neck or a pair of hooks to partially enclose the neck) that is configured to engage a neck of the user. In some embodiments, the neck support 104 engages a region of the upper chest and/or shoulders. In an embodiment, the apparatus 100 also includes a housing 106 that defines an interior space. Upon engaging the neck of the user with the neck support 104, the housing 106 is positioned adjacent a base of a neck and/or a collar bone of the user.

In an embodiment, the apparatus 100 includes a sanitary barrier, such as a mask sheet 110. In one embodiment, the housing 106 is configured to receive the mask sheet 110 such that the mask sheet 110 is movable from a first position or extended position 103 (e.g., right side of FIG. 2A) where the mask sheet 110 covers a portion of a face (e.g. nose and mouth) to a second position or retracted position 102 (e.g. left side of FIG. 2A) where the mask sheet 110 is positioned within the housing 106. In an example embodiment, in the extended position 103 the mask sheet is configured to reduce transmission of pathogens between the face of the user and surroundings of the user. In one embodiment, the apparatus includes a pair of ear loops 108a, 108b on opposite sides of the mask sheet 110 that are configured to hook around the respective ears of the user (right side of FIG. 2A). In one example embodiment, the ear loops 108′ are detachable from the mask sheet 110 (FIG. 2G).

In one embodiment, the mask sheet 110 is movable from the extended position 103 to the retracted position 102. In an example embodiment, the mask sheet 110 is movable from the extended position 103 to the retracted position 102 by rotating and/or curling up within the housing 106. FIG. 2B is an image that illustrates an example of the mask sheet 110 of the apparatus 100 wrapped around an inner rod 130 that is rotatably coupled within the housing 106, according to an embodiment. In one embodiment, the inner rod 130 is pivotally coupled within the housing 106. In one example embodiment, the inner rod 130 is pivotally coupled to a spring-loaded ratchet gear 114 within the housing 106. In one example embodiment, the apparatus 100 includes a pawl 112 to engage the ratchet gear 114 and prevent further rotation of the ratchet gear 114 (and inner rod 130) upon engaging one or more teeth of the ratchet gear 114. In an example embodiment, the pawl 112 engages the ratchet gear 114 when the mask sheet 110 is moved to the extended position 103, so the ratchet gear 114 does not pivot the inner rod 130 and cause the mask sheet 110 to move back to the retracted position 102 within the housing 106.

In an embodiment, a switch (not shown) is provided on the housing 106 and can be pressed by the user (e.g., when the user no longer needs the mask sheet 110 in the extended position 103) to cause the mask sheet 110 to move to the retracted position 102. In an example embodiment, the switch is operatively coupled to the pawl 112 such that pressing the switch causes the pawl 112 to disengage the ratchet gear 114, the ratchet gear 114 to subsequently cause the inner rod 130 to pivot and the mask sheet 110 to move from the extended position 103 to the retracted position 102. In another example embodiment, the inner rod 130 features one or more prongs at opposite ends of the inner rod 130 which are configured to be received within one or more slots on an inner surface of the ratchet gear 114. Although some embodiments discuss the mask sheet 110 being rotated within the housing 106 in the retracted position 102, in other embodiments the mask sheet 110 can be translated to the retracted position 102. In this example embodiment, the housing 106 is a rectangular or planar shaped housing with a slot to receive the mask sheet translating into the housing from the extended position 103 to the retracted position 102).

In one embodiment, as the mask sheet 110 is moved from the extended position 103 (e.g., right side of FIG. 2A) to the retracted position 102 (e.g. left side of FIG. 2A) the mask sheet 110 is received within a slot (not shown) on the housing 106 and wraps around the inner rod 130 within the housing 106, as the inner rod 130 pivots within the housing 106 (e.g. along with the ratchet gear 114). In an example embodiment, the inner rod 130 and/or the mask sheet 110 are replaceable (e.g., as a cartridge) by opening the housing 106 (e.g. removing a portion of the housing 106 such that the inner rod 130 and/or mask sheet 110 can be removed and replaced). This advantageously permits the user to replace the inner rod 130 and/or mask sheet 110 without requiring purchase of a new housing 106.

In one example embodiment, the circumference of the inner rod 130 is based on a length of the mask sheet 110 (e.g., where the length of the mask sheet 110 is defined as a dimension of the mask sheet 110 that extends from the housing 106 to a top region of the mask 110 in the extended position 103 and/or a dimension of the mask sheet 110 that covers the nose and mouth of the user). In one example embodiment, the circumference of the inner rod 130 is about equal (e.g., within ±20%) of the length of the mask sheet 110 so that the mask sheet 110 does not self-overlap when it wraps around the inner rod 130 as it moves from the first position 103 to the second position 102.

In an embodiment, the apparatus 100 includes a radiation source or light source mounted within the interior space of the housing 106 and is configured to emit radiation to sterilize the mask sheet 110 in the retracted position 102. In one embodiment, the light source is an ultraviolet (UV) light source. In an example embodiment, the light source is a UV light emitting diode (LED) panel 120 arranged within the interior space of the housing 106 such that UV light 121 (FIG. 2C) emitted from the UV LED panel 120 is directed at the mask sheet 110. In an example embodiment, the wavelength of the UV light 121 is in a range from about 270 nanometers (nm) to about 275 nm and/or in a range from about 220 nm to about 300 nm and/or in a range from about 200 nm to about 400 nm. In one example embodiment, the UV LED panel 120 is arranged around an inner circumference of the housing 106 (e.g., between the inner rod 130 and an inner surface of the housing 106). In one example embodiment, the UV LED panel 120 is arranged around the entire inner circumference of the housing 106. In the embodiments of the present invention, light 121 (e.g. ultraviolet C radiation) is used to kill bacteria and viruses on the mask sheet 110. Several factors were considered in the design of the apparatus 100. In one embodiment, in order to provide maximum exposure (e.g., UVC exposure) to the mask sheet 110 and limit exposure to the wearer, the light-emitting diode (LED) panel 120 is contained within the housing 106 with the mask sheet 110 at the time of sterilization. To achieve this, the housing 106 was designed to be worn around the wearer's neck, with the mask sheet 110 contained inside (e.g. in the retracted position 102) while not being used. Also, in this embodiment, the housing 106 is made from material that is highly absorbent of the UV light 121, to minimize the extent of UV light 121 escaping the housing 106 and thus limiting exposure to the wearer.

In one embodiment, when the mask sheet 110 is moved from the extended position 103 to the retracted position 102, the circumference of the inner rod 130 is sufficiently large that the mask sheet 110 does not self-overlap on the inner rod 130 in the retracted position 102. This arrangement advantageously ensures that the UV light 121 emitted from the UV LED panel 120 only needs to penetrate a single layer of the mask sheet 110 to sterilize the mask sheet 110.

In another embodiment, as the mask sheet 110 moves from the extended position 103 to the retracted position 102, an outer surface of the mask sheet 110 (e.g., oriented at the surroundings of the user in the extended position 103) is directed in a radially outward direction when the mask sheet 110 is wrapped around the inner rod 130 in the retracted position 102. This advantageously ensures that the UV light 121 from the UV LED panel 120 (e.g., directed in a radially inward direction within the housing 106) is directed at the outer surface of the mask sheet 110 (e.g. where a normal to the outer surface of the mask sheet 110 is directed in the radially outward direction within the housing 106). Thus, this arrangement increases the sterilization efficiency of the outer surface of the mask sheet 110. In an example embodiment, the outer surface of the mask sheet 110 is more likely to contain pathogens (e.g., from external source and/or from the user touching the outer surface of the mask sheet 110 in the extended position 103, etc.). However, in other embodiments, even when the outer surface of the mask sheet 110 is oriented in the radially outward direction within the housing 106, the UV light 121 is still configured to sterilize both the inner surface and outer surface of the mask sheet 110. In still other embodiments, UV light sources (e.g. UV LED panel 120) may be positioned within the inner rod 130 and direct UV light 121 in a radially outward direction, to sterilize the inner surface of the mask sheet 110 (e.g. the surface of the mask sheet 110 oriented at the wearer in the extended position 103).

FIG. 2D is a block diagram that illustrates an example of the apparatus 100 of FIGS. 2A and 2B, according to an embodiment. For purposes of FIG. 2D, thin lines (1.5 point) are depicted to indicate physical or optical coupling between components of the apparatus 100 and thick lines (3.5 point) are depicted to indicate communication signals between components of the apparatus 100. In an embodiment, FIG. 2D depicts the switch 113 of the apparatus 100 that is pressed by the user to cause the mask sheet 110 to move from the extended position 103 to the retracted position 102. As shown in FIG. 2D, the switch 113 is operatively coupled to the pawl 112 such that upon pressing the switch 113, the pawl 112 disengages the ratchet gear 114 and consequently the ratchet gear 114 rotates the inner rod 130 within the housing 106 and moves the mask sheet 110 to the retracted position 102.

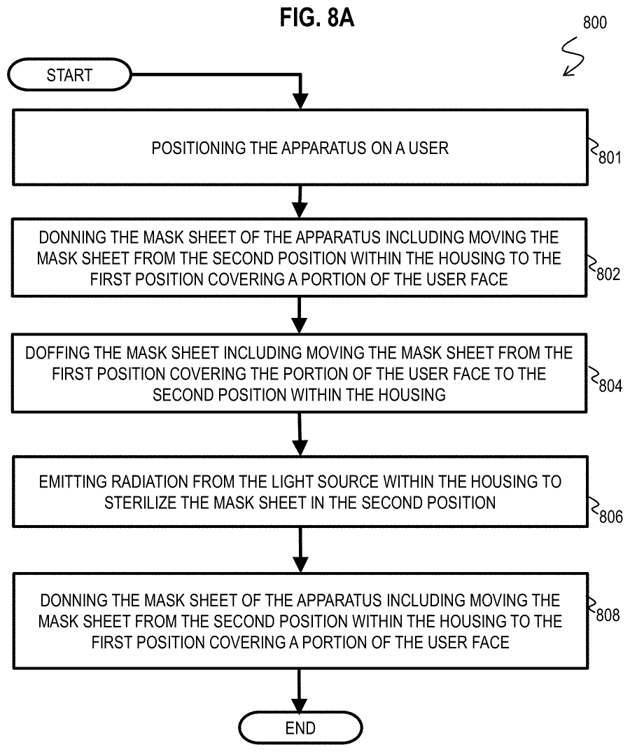

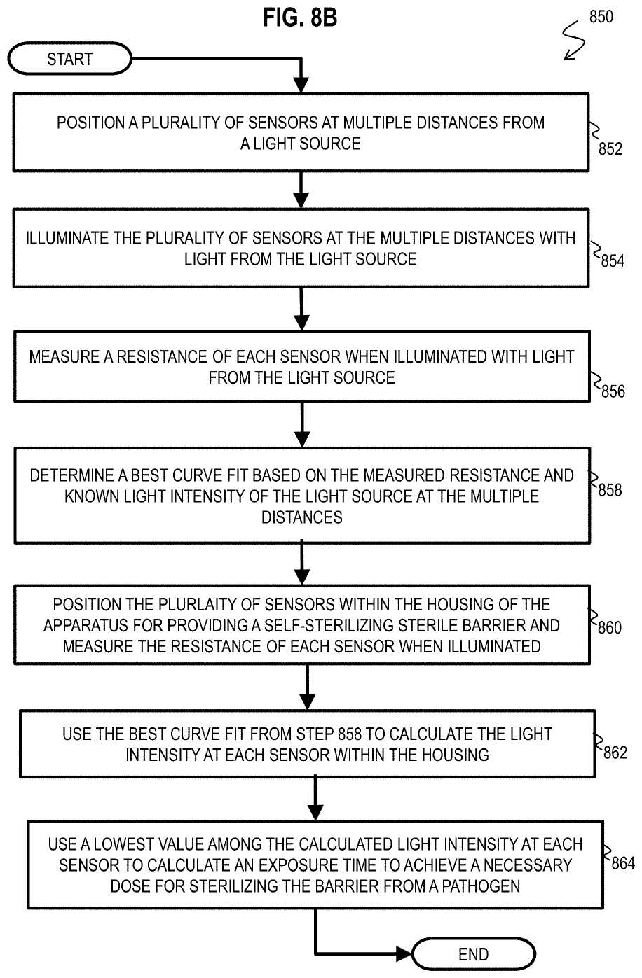

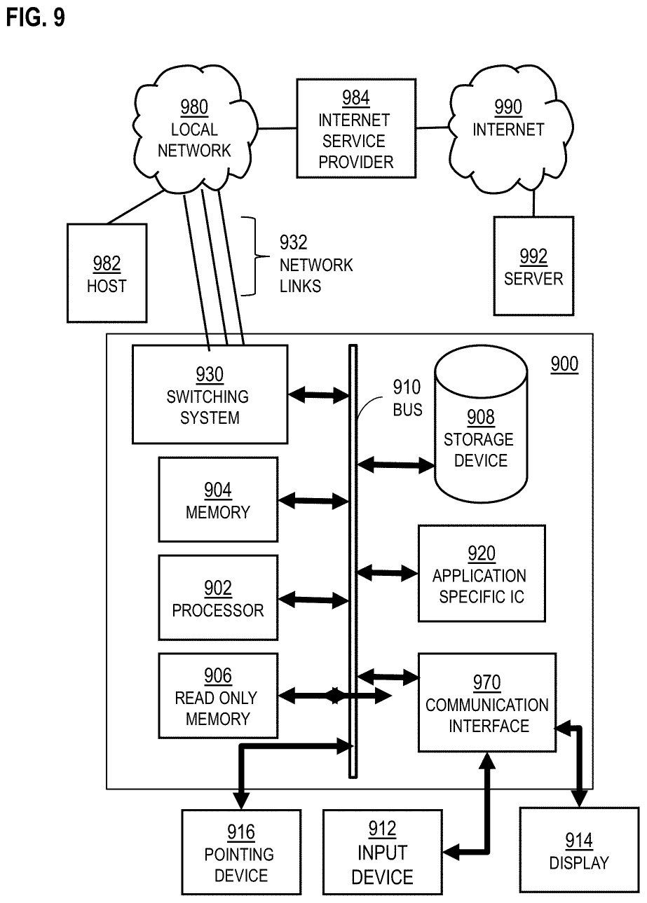

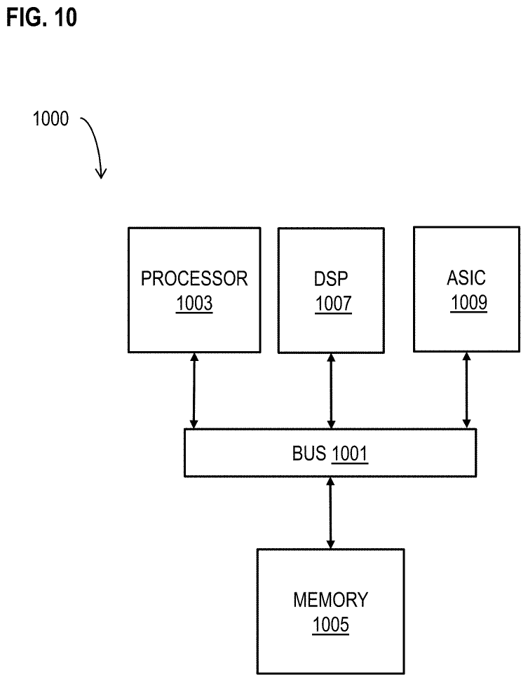

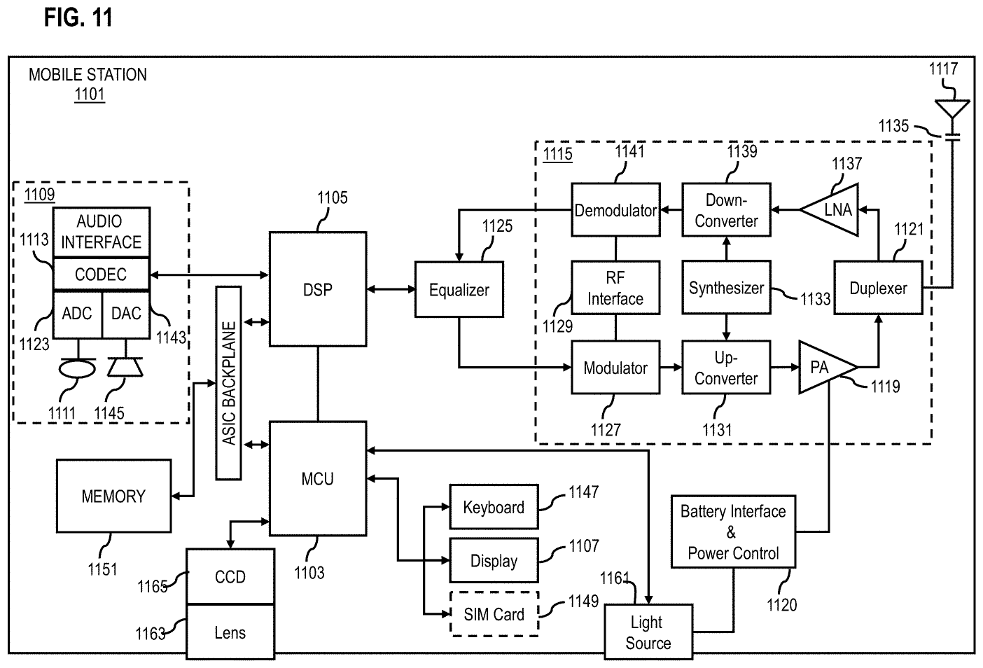

In one embodiment, as shown in FIG. 2D the apparatus 100 includes a controller 101, such as a computer system described below with reference to FIG. 9, a chip set described below with reference to FIG. 10 and/or a mobile terminal (e.g., smart phone) described below with reference to FIG. 11. A memory 105 of the controller 101 includes a sterilization module 109 with instructions to perform one or more steps of the method 800 based on the flowchart of FIG. 8A or the method 850 based on the flowchart of FIG. 8B.

In an embodiment, the apparatus 100 includes a sensor 107 that is communicatively coupled with the controller 101. In an example embodiment, the sensor 107 is configured to detect when the mask sheet 110 has moved from the extended position 103 to the retracted position 102. The sensor 107 transmits a signal to the controller 101 upon detecting that the mask sheet 110 has moved to the retracted position 102. In an embodiment, upon receiving the signal from the sensor 107, the controller 101 transmits a signal to the UV LED panel 120 to cause the UV LED panel 120 to emit UV light 121 at the mask sheet 110 to sterilize the mask sheet 110. In an example embodiment, the mask sheet 110 is sterilized based on a minimum dosage of UV light 121, defined as:

UV dose=UV intensity×Exposure time (1)

where UV dose is the dosage of UV light (in units of μW·s/cm²), UV intensity is the intensity of the UV light 121 (in units of μW/cm²) incident on the mask sheet 110 and exposure time is the time (in units of seconds) that the mask sheet 110 is exposed to the UV light 121 with the UV intensity. In an embodiment, equation 1 is stored in the memory 105 of the controller 101 and is used to determine whether a particular intensity of UV light 121 at a specific exposure time will achieve a minimum dosage of UV light 121. In one example embodiment, a minimum dosage of UV light in a range from about 2,000 to about 8,000 μW·s/cm² is used to sterilize the mask sheet 110. In an example embodiment, the minimum dosage of UV light to sterilize the mask sheet 110 is stored in the memory 105 of the controller 101. In an example embodiment, to determine the exposure time for a particular UV LED panel 120, the intensity value of the UV LED panel 120 and the minimum dosage value (e.g. within the above range) are input into equation 1 which is subsequently solved to determine the value of the exposure time. In an example embodiment, the user can input with an input device (e.g., keyboard 1147 or touch screen display 1107 of the mobile terminal 1101 in FIG. 11, or the input device 912 or pointing device 916 of the computer system 900 in FIG. 9) a desired value for one or more parameters of equation 1. In an example embodiment, the user inputs the exposure time and/or UV intensity into the controller 101. If the user inputs a desired exposure time value, the controller 101 uses the inputted exposure time and the known minimum UV dosage to sterilize the mask with equation 1 to calculate the UV intensity to achieve the minimum dosage to sterilize the mask sheet. If the user inputs a desired UV intensity value and/or a desired UV dose value to achieve sterilization, the controller 101 uses the inputted values with equation 1 to calculate the exposure time to achieve the minimum dosage to sterilize the mask sheet.

In one embodiment, after transmitting the signal to the UV LED panel 120 to emit the UV light 121, the controller 101 transmits a subsequent signal to the UV LED panel 120 after the minimum time period has elapsed, to deactivate the UV LED panel 120. This advantageously ensures that the mask sheet 110 is sterilized while conserving energy. In an embodiment, a power source (not shown) is provided within the housing 106 and is electrically coupled to one or more of the sensor 107, the controller 101 and/or the UV LED panel 120. In other embodiments, one or more of the sensor 107, the controller 101 and the UV LED panel 120 have an internal power source. In an example embodiment, the power source is a battery that is rechargeable (e.g., using the USB-C port 122 in FIG. 2B or an electrical outlet).

FIG. 2E is an image that illustrates an example of a side perspective view of the mask sheet 110, the inner rod 130 and the housing 106 of the apparatus 100, according to an embodiment. FIGS. 2F through 2H are images that illustrate an example of accessories to be used with the apparatus 100 of FIGS. 2A and 2B, according to an embodiment. In an embodiment, FIG. 2F depicts a support (e.g., soft support) along a top of the mask sheet 110, to adapt to the contour of the user's face and thus provide a closer fit to the face of the user (e.g. to minimize passage of pathogens between the mask sheet and the face of the user). FIG. 2H depicts a hook member of the neck support 104 which is configured to engage a second hook (e.g., on the opposite side of the neck support 104) behind a neck of the user, to securely attach the apparatus 100 to the user.

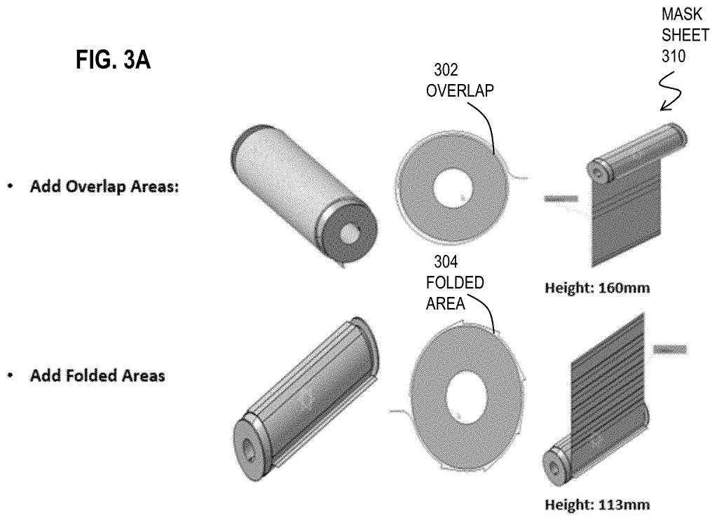

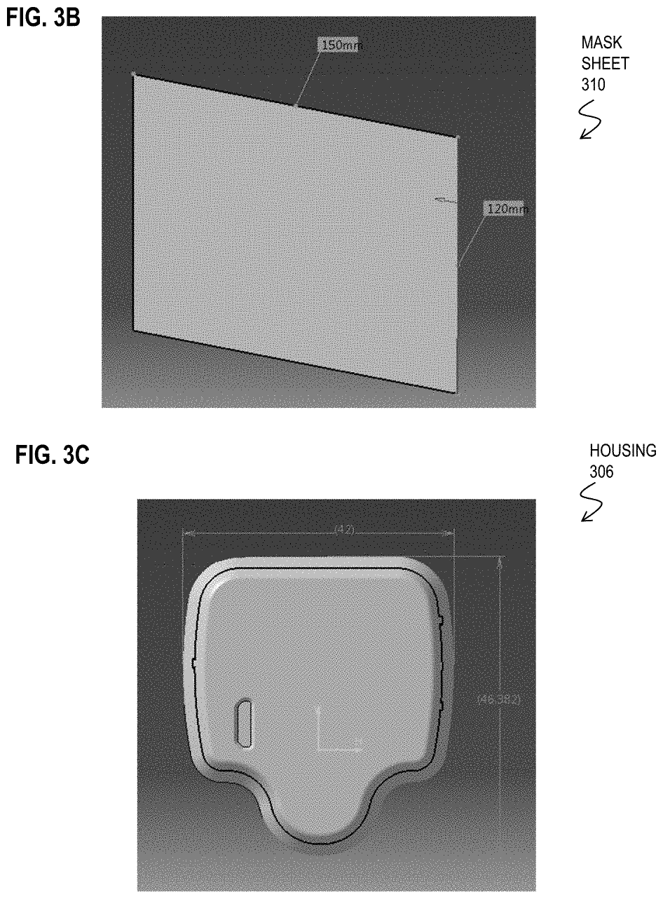

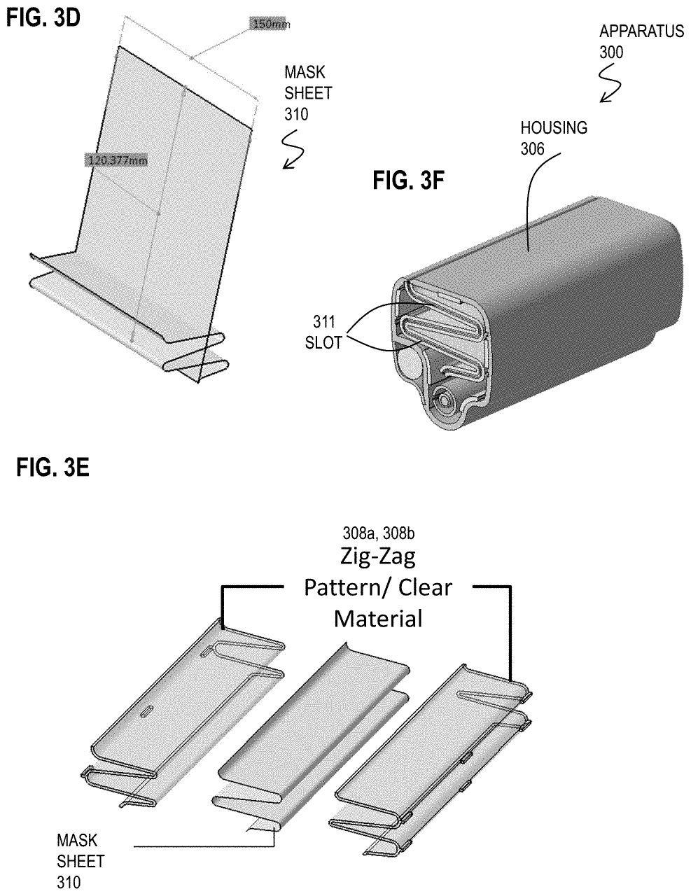

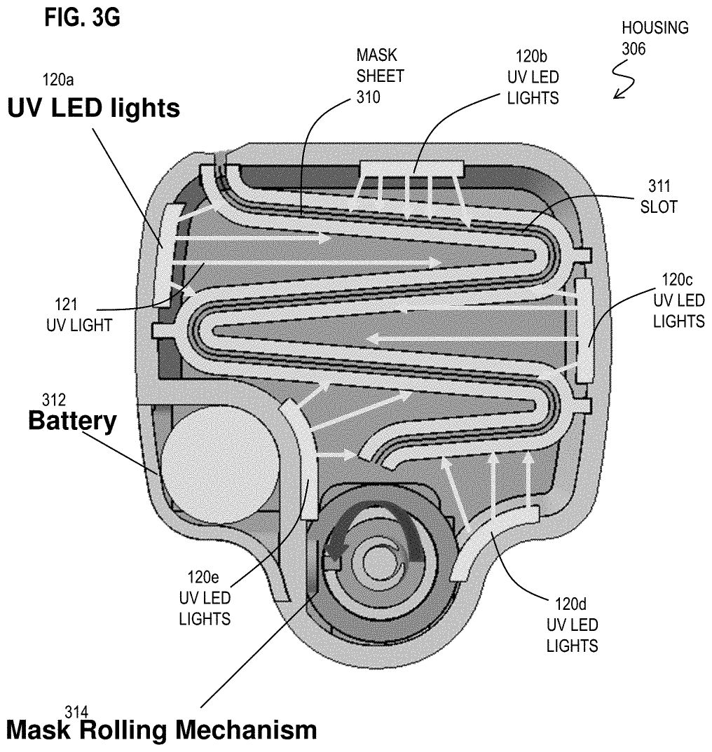

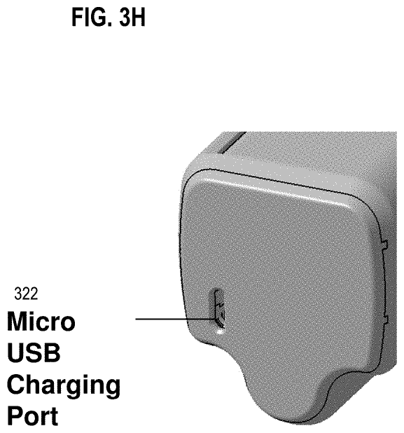

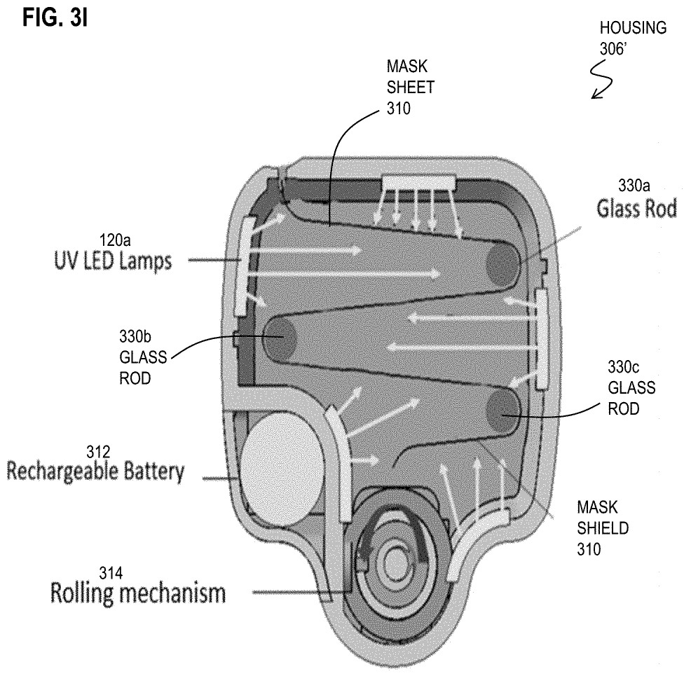

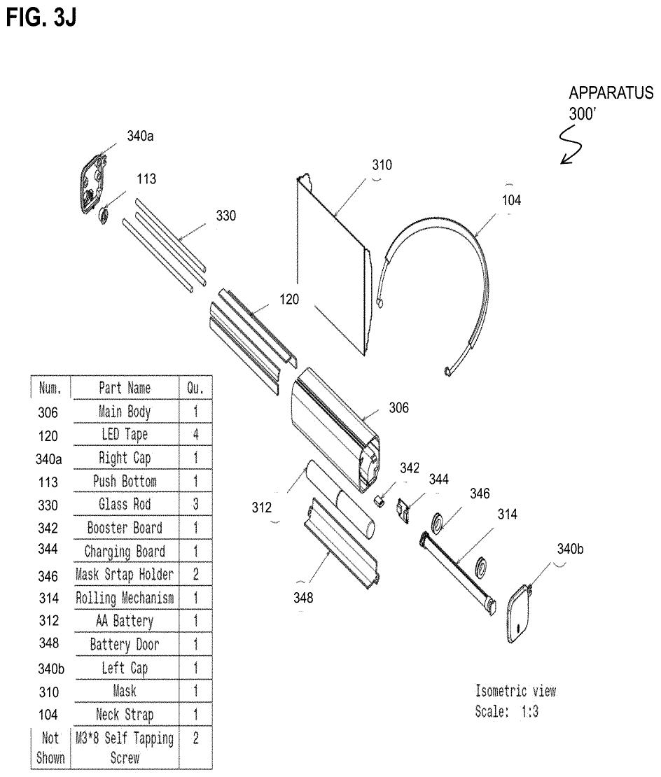

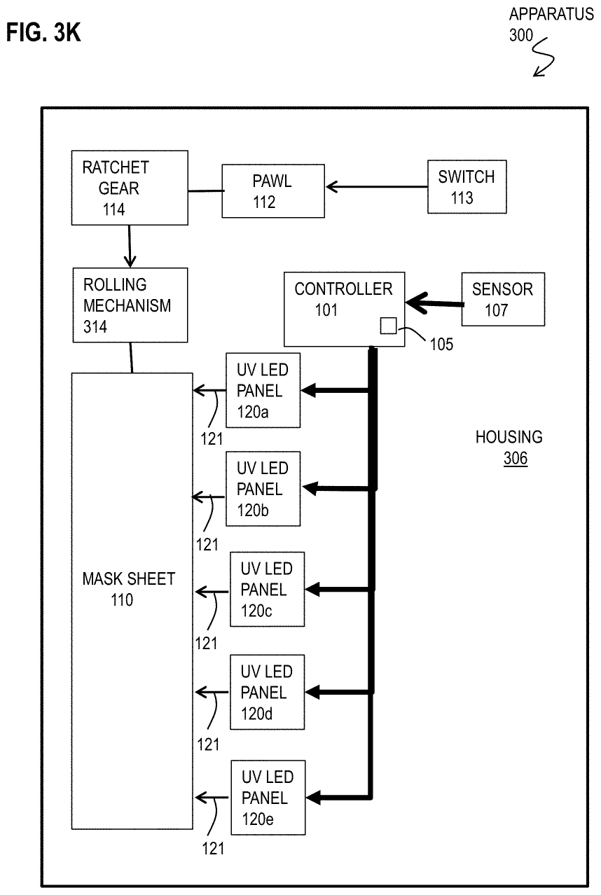

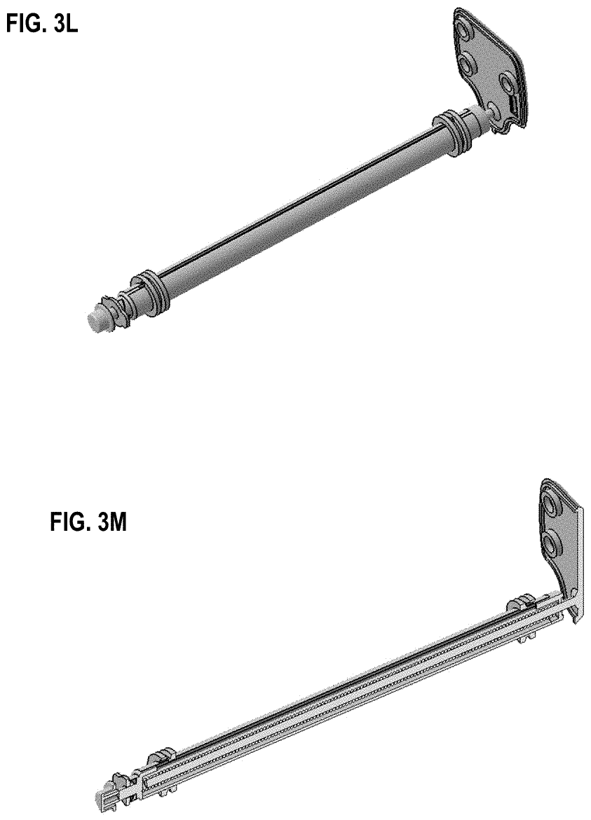

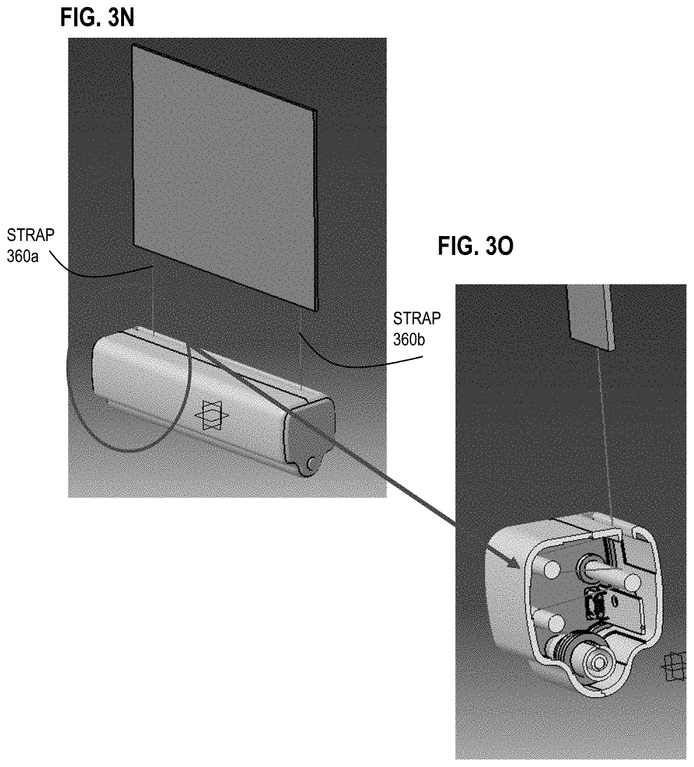

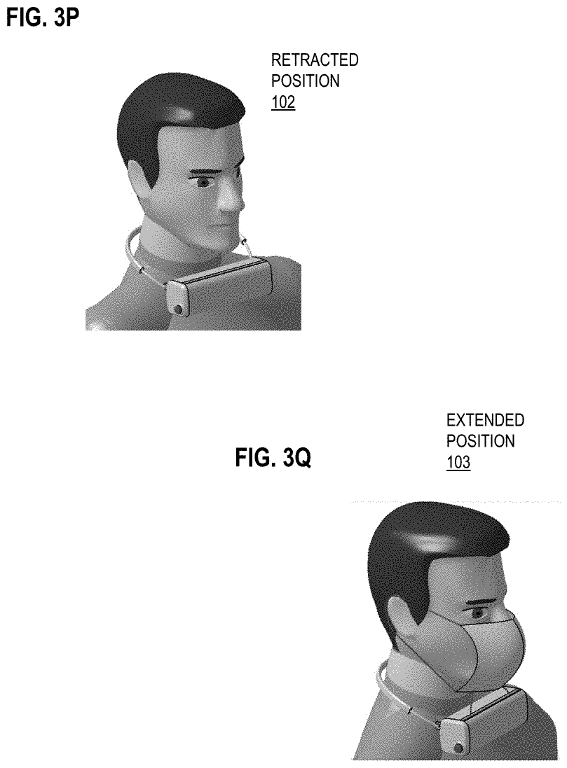

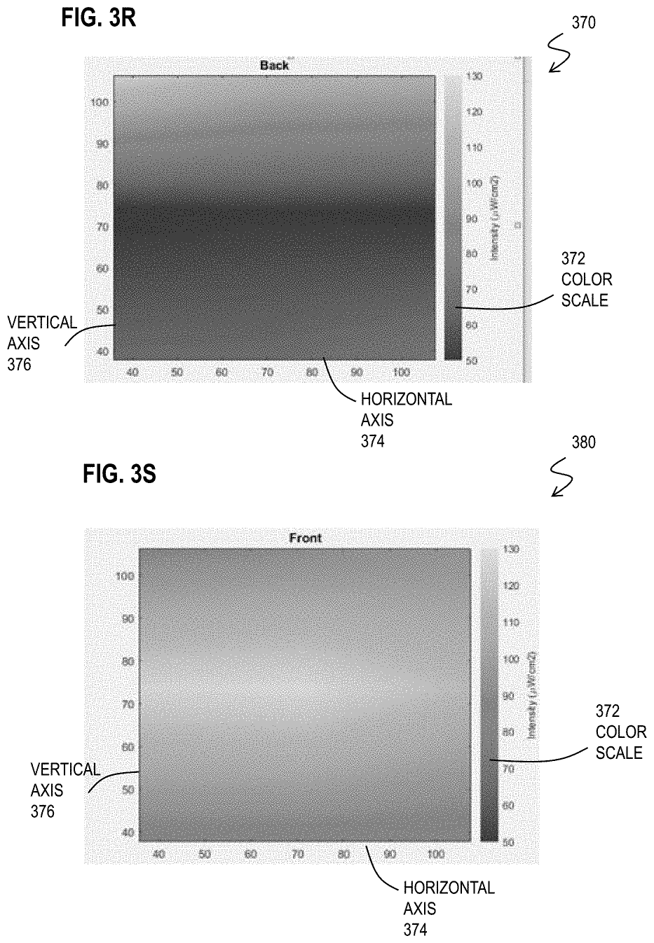

FIG. 3A is an image that illustrates an example of an overlap or folded area in the mask sheet wrapped around the inner rod 130 of the apparatus of FIG. 2B. In an embodiment, the inventors of the present invention recognized that the apparatus 100 could be improved if a larger mask sheet 310 was utilized. In one example embodiment, the larger mask sheet 310 (FIG. 3B) has a width of about 150 millimeters (mm) or in a range from about 120 mm to about 180 mm and a height of about 120 mm or in a range from about 100 mm to about 140 mm. In an example embodiment, the inventors of the present invention selected the range of the mask sheet 310 based on using 3D software (e.g. CATIA®) with models of different genders and/or ethnicities to ensure the selected range covers a large segment of the population. To accommodate the larger mask sheet 310, the mask sheet 310 could be wound around the inner rod 130 so that an overlap 302 (FIG. 3A) in the mask sheet is provided. Another alternative is if folded areas 160 are provided in the mask sheet 310 and the folded areas 160 are wound around the inner rod 130 (FIG. 3B).

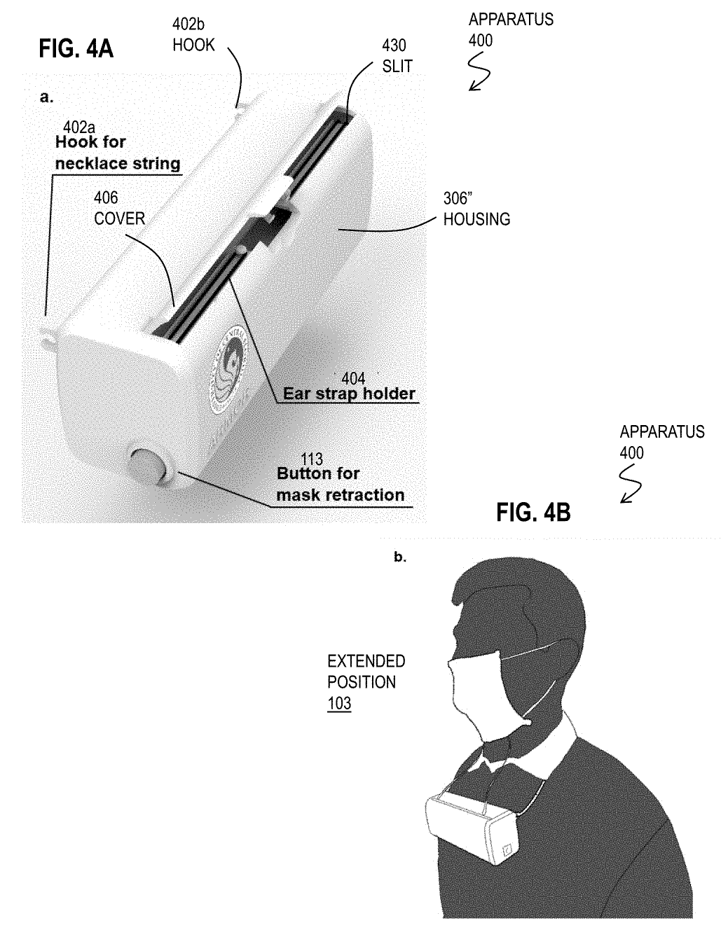

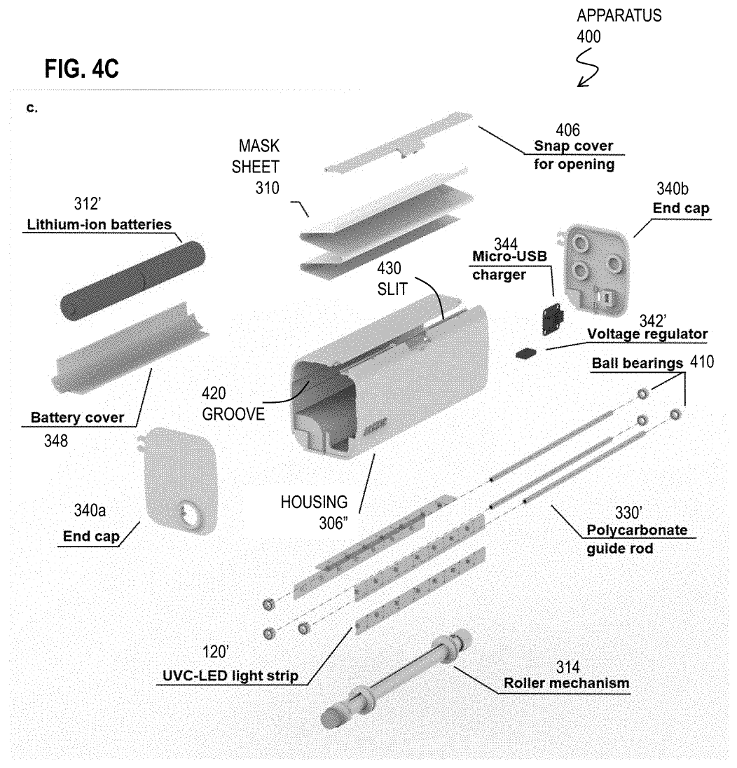

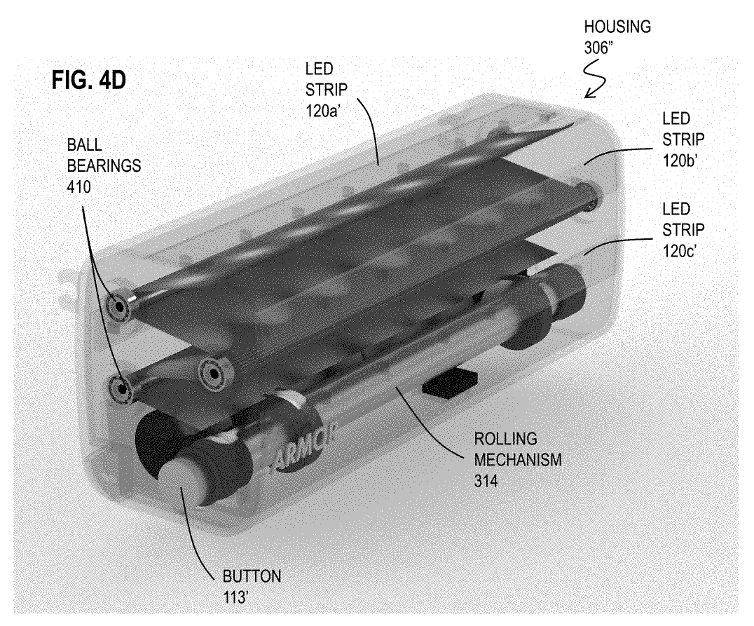

However, the inventors recognized that these designs of FIGS. 3A and 3B may pose an issue in terms of sterilization, since they may require the UV light 121 to pass through multiple layers of the mask sheet 310 for sterilization and/or require the inner rod 130 diameter (and hence the housing 106) to be undesirably enlarged. Thus, the inventors of the present invention developed an improved design to accommodate the larger mask sheet 310 with an improved housing 306 (FIG. 3C). This improved design for the housing 306 ensures that the larger mask sheet 310 is sterilized. One embodiment for the improved housing 306 is discussed below with respect to FIGS. 3D through 3H. Another embodiment for the improved housing 306′ is discussed below with respect to FIGS. 3I and 3J. Yet another embodiment for the improved housing 306″ is discussed below with respect to FIGS. 4A through 4D.

FIGS. 3D through 3H are images that illustrate an example of various views of an apparatus 300 for providing a self-sterilizing sanitary barrier, according to an embodiment. The apparatus 300 is similar to the apparatus 100 discussed previously, with the exception of the features discussed herein. In an embodiment, the housing 306 defines an interior space configured to receive the mask sheet 310 as the mask sheet 310 is moved from the extended position 103 (FIG. 3Q) to the retracted position 102 (FIG. 3P) within the housing 306. In an embodiment, as shown in FIG. 3C, the housing 306 has reduced dimensions relative to the housing 106, specifically a width of about 42 mm or in a range from about 30 mm to about 50 mm and a height of about 46 mm or in a range from about 35 mm to about 55 mm. The housing 306 is configured so that the mask sheet 310 takes an arcuate path within the interior space of the housing 306 as the mask sheet 310 moves from the extended position 103 to the retracted position 102. In one embodiment, the arcuate path is a non-circular path, such as a zig-zag path or a tortuous path or a serpentine path defined by a plurality of turns within the interior space. However, the embodiments of the invention are not limited to the arcuate path being a serpentine path and include any arcuate path within the interior space (e.g., an arced path that does not include multiple turns, an arced path that is non-circular, etc.).

As shown in FIGS. 3E and 3F, in one embodiment, the serpentine path is defined by a slot 311 that is formed between two spaced apart layers 308a, 308b. As the mask sheet 310 moves from the extended position 103 to the retracted position 102, the mask sheet 310 moves into the interior space of the housing 306 and along the slot 311 defined between the two spaced apart layers 308a, 308b. In an embodiment, the layers 308a, 308b of material each have a serpentine shape such that the slot 311 between them also has the serpentine shape.

As shown in FIG. 3G, in one embodiment the interior space of the housing 306 includes a plurality of light sources. In one embodiment, the light sources are a plurality of UV light sources (e.g., UV LED light panels 120a through 120d) that are mounted at different locations within the interior space of the housing 306. In one embodiment, the quantity and location of the UV LED panels 120 used is selected so to ensure that a threshold intensity of UV light (e.g., based on equation 1) reaches each segment of the mask sheet 310. In another embodiment, the quantity and locations of the UV LED panels 120 mounted within the interior space is selected such that the UV light 121 from a UV LED panel 120 is incident on each segment of the mask sheet 310 (e.g., without having to pass through one or more layers of the mask sheet 310 prior to reaching that segment). As shown in FIG. 3G, the quantity and location of the UV LED panels 120a through 120d direct UV light 121 at each segment of the mask sheet 310 without having to pass through multiple layers of the mask sheet 310. Although FIG. 3G depicts five UV LED panels mounted at distinct locations within the housing 306, the embodiments of the present invention is not limited to this arrangement and includes less or more than five UV LED panels mounted in the same of different locations, depending on various factors including dimensions of the housing, dimensions of the mask sheet, number of turns in the slot 311, etc., such that each segment of the mask sheet receives the threshold intensity of UV light for sterilization.

In an embodiment, the housing 306 includes a mask rolling mechanism 314 that is operated similar to the inner rod 130, ratchet gear 114 and pawl 112 of the apparatus 100. In an embodiment, one exception in the operation of the mask rolling mechanism 314 relative to the inner rod 130 of the apparatus 100 is that the mask sheet 310 does not wrap around the mask rolling mechanism 314. Instead, a pair of straps 360a, 360b (FIG. 3N) wrap around the mask rolling mechanism 314 as the mask sheet 310 moves from the extended position 103 to the retracted position 102. This advantageously ensures that the mask sheet 310 is within the serpentine slot 311 (above the mask rolling mechanism 314) where the mask sheet 310 is exposed to the UV light 121 from the multiple UV LED panels 120a through 120d. As shown in FIG. 3O, in the extended position 103, the straps 360a, 360b are within the serpentine shaped slot 311 of the housing 306. As the mask sheet 310 moves from the extended position 103 to the retracted position 102, the straps 360a, 360b wrap around the mask rolling mechanism 314 (e.g. positioned in a bottom region of the interior space of the housing 306) and the mask sheet 310 is received within the serpentine slot 311 between the layers 308a, 308b.

As shown in FIG. 3G, in one embodiment the interior space of the housing 306 includes a power source (e.g., battery 312) for one or more of the LED panels 120a through 120d. In one embodiment, where the housing 306 includes the sensor 107 and controller 101 that is included in the housing 106, the battery 312 is also a power source for the sensor 107 and/or the controller 101. In other embodiments, the controller 101 is located external to the housing 306 (e.g., mobile station 1101 of FIG. 11). As shown in FIG. 3H, in one embodiment, the housing 306 includes a charging port 322 (e.g., micro USB) to conveniently recharge the battery 312.

Another design for the housing is now discussed with respect to FIGS. 3I and 3J. As with the housing of FIGS. 3D through 3G, the housing 306′ is configured such that the mask sheet 310 takes an arcuate path (e.g. a serpentine path) as the mask sheet 310 moves from the extended position 103 to the retracted position 102. However, the housing 306′ differs from the housing 306 of FIGS. 3D through 3G in terms of the structure that facilitates the mask sheet 310 moving along the serpentine path.

In an embodiment, the housing 306′ of FIGS. 3I and 3J is similar to the housing 306 discussed above, with the exception of the features discussed herein. In one embodiment, as with the housing 306, the housing 306′ is similarly configured so that the mask sheet 310 takes an arcuate path as the mask sheet 310 is moved from the extended position 103 to the retracted position 102. Unlike the housing 306, where the serpentine path is provided by a pair of spaced apart layers 308a, 308b, the housing 306′ provides a serpentine path based on a plurality of rods or rollers that are mounted within the interior space of the housing 306′. As shown in FIG. 3I, the housing 306′ includes a plurality of rods 330a through 330c over which the mask sheet 310 is configured to move in the serpentine path (e.g., a plurality of turns). In an example embodiment, the rods 330a through 330c are made from a material (e.g., glass) that is transmissive to the radiation from the light source (e.g., UV light 121). This advantageously ensures that the rods 330a through 330c do not interfere with the sterilization process where a threshold intensity of UV light 121 is incident on the mask sheet 310. In one embodiment, the rollers 330a through 330c are rotatably mounted within the interior space of the housing 306′ such that the rollers 330a through 330c collectively rotate as the mask sheet 310 moves from the extended position 103 to the retracted position 102 within the housing 306′ along the serpentine path. Although three rollers 330a through 330c mounted in distinct locations are depicted in FIG. 3I, less or more than three rollers in the same or different mounting locations can be utilized based on various parameters (e.g. a dimension of the mask sheet 310, a dimension of the housing 306′, a number of turns in the serpentine path, etc.) such that the mask sheet 310 takes the serpentine path over the rollers.

In an embodiment, when the mask sheet 310 moves to the retracted position 102 (e.g., is collected into the housing 306′), the arrays of UV LED panels 120a through 120d turn on and start the disinfection process of the mask sheet 310. In an example embodiment, the housing 306 includes the sensor 107 that detects when the mask sheet 310 has moved to the retracted position 102 and transmits a signal to the controller 101 to initiate the sterilization process. In the example embodiment of FIG. 3I, five arrays or LED panels 120a through 120e are used, to provide enough dosage of UV light 121 on the mask sheet 310. This advantageously increases the efficiency of sterilizing the mask sheet 310. In an example embodiment, the serpentine or zigzag orientation of the mask sheet 310 around the glass rods 330a through 330c allows the entire mask sheet 310, including the front and back sides, to be exposed to the UV light 121. In an embodiment, since UV light 121 can negatively impact to the skin and eyes when directly exposed, the housing 306′ is made with a material that is not transmissive of UV light and thus prevents unwanted exposure.

FIG. 3J depicts an exploded view of the housing 306′ of FIG. 3I. In an embodiment, the UV LED panels 120a through 120e require a certain threshold voltage (e.g., 12 volts) from the battery 312 to produce proper wavelengths (e.g. in a range from about 270 nanometers or nm to about 275 nm and/or in a range from about 220 nm to about 300 nm) that can sterilize pathogens (e.g. bacteria and/or viruses). In one embodiment, the inventors selected a widely available rechargeable battery 312 for use with the apparatus 300 that has a low voltage (e.g., about 3.7 volts) lower than the threshold voltage for the UV LED panels. In one embodiment, to control the consistent intensity of the UV LED panels 120, a booster circuit 342 is provided to increase the voltage of two rechargeable batteries 312. In an example embodiment, the booster circuit 342 harnesses with a battery management board 344 that can charge the batteries 312. In another embodiment, the booster circuit 342 and/or battery management board 344 are wirelessly connected to the controller 101 that is used to control the operation of the apparatus 300′. The controller 101 is similar to the controller 101 of the apparatus 100 and in one embodiment includes the mobile station 1101 (e.g., smart phone) of FIG. 11. In one embodiment, the booster circuit 342 and/or the battery management board 344 are in wireless communication (e.g., Bluetooth® connection) with the controller 101 (e.g. mobile station 1101). In one example embodiment, a user can observe and adjust the exposure time (expressed in equation 1) with the mobile station 1101 (e.g., with the keyboard 1147 and/or touch screen 1107) using software (e.g. an app) that is installed on the smart phone.

In one embodiment, as with the mask inner rod 130, pawl 112 and ratchet gear 114 of the apparatus 100, the rolling mechanism 314 uses a coiled tensile spring to manage a large number of rotations when the mask sheet 310 is being pulled out of (unwinding the spring) and into the (winding the spring) the housing 306′. When the mask sheet 310 is pulled out to the extended position 103, the ratchet gear 114 of the rolling mechanism 314 holds the position of the mask sheet 310. To move the mask sheet 310 into the housing 306′, the user presses a button (e.g., similar to the switch 113 of the apparatus 100) to disengage the ratchet gear 114. In an example embodiment, as with the apparatus 100, the housing 306′ will hang around the neck with the neck support 104 and is designed in such a way that users can detach the neck support 104 from the mask.

Another design for the housing is now discussed with respect to FIGS. 4A through 4D. The housing 306″ of FIGS. 4A through 4D is similar to the housing 306′ with the exception of the features discussed herein. In one embodiment, the housing 306″ defines a pair of hooks 402a, 402b to attach the housing 306″ to the neck support 104. In one embodiment, the housing 306″ defines a slot 430 through which the mask sheet 310 is passed as it moves from the extended position 103 (FIG. 4B) to the retracted position 102. To minimize radiation exposure of the wearer and the surroundings, the housing 306″ includes a cover 406 that covers the slit 430 when the mask sheet 310 is moved to the retracted position 102. In one embodiment, the cover 406 is movable from a first position (not covering the slit 430) to a second position (covering the slit 430). In an example embodiment, the cover 406 is rotatably attached to the housing 306″ to rotate from the first position (FIG. 4A) to the second position covering the slit 430. In an example embodiment, the cover 406 is a snap-in-place cover is used to close off the slit 430 at the top of the housing 306″ in order to reduce the risk of UV exposure and prevent dust from entering the interior space (e.g., sanitization chamber) within the housing 306″ when the mask sheet is within the interior space (e.g. retracted position 102 in FIG. 3P). In an example embodiment, the cover 406 is made from material that is highly absorbent of UV radiation (e.g., same material as the housing 306″).

In an embodiment, underneath the cover 406 is an ear strap holder 404 to hold the ear loops 108a, 108b in place while the mask sheet 310 is in the housing 306″ in the retracted position 102. In one embodiment, the ear strap holder 404 includes a tray with two hooks designed to hold the ear loops 108a, 108b in place while the mask sheet 310 is in the housing 306″. When the user wants to use the mask sheet 310, the cover 406 can be opened, to deploy the ear loops 108a, 108b of the mask sheet 310 for the user to grab so the mask sheet 310 can be safely and easily extracted from the housing 306″ without contamination and worn for as long as necessary (e.g. in the extended position 103 in FIG. 4B). If the mask sheet 310 becomes contaminated, it can be retracted back into the housing 306″ with a simple press of the button 113 and the ear loops 108a, 108b can be hooked back in place on the ear strap holder 404. The mask sheet 310 is then disinfected.

FIG. 4C is an image that illustrate an example of an exploded view of the apparatus 400 of FIG. 4A including the housing 306″, according to an embodiment. FIG. 4D is an image that illustrates an example of a perspective view of the housing 306″ of FIGS. 4A through 4C, according to an embodiment. In an embodiment, the exploded view of the apparatus 400 of FIG. 4C is similar to the exploded view of the apparatus 300′ in FIG. 3J, with the exception of the features discussed herein.

In designing the housing 306″, a thickness of the housing 306″ is selected to protect the wearer against UV exposure. The penetration depth of UV light in most polymers is between about 0.025 mm and about 0.05 mm. [18] Thus, in one embodiment, the thickness of the housing 306″ is selected to be greater than this penetration depth. In an example embodiment, the thickness of the housing 306″ case is about 2 mm or in a range from about 0.5 mm to about 4 mm. In this example embodiment, the thickness of the housing 306″ is selected for all areas except grooves 420 (FIG. 4C) where the UV light strips 120′ are positioned. In this example embodiment, the thickness of the housing 306″ along the grooves 420 are less (e.g. about 1.3 mm) than the rest of the housing 306″. In one embodiment, the groove 420 is sized so to receive the LED light strip 120′ (e.g. so the LED light strip 120′ is slidable into the groove 420 or snaps into the groove 420). This advantageously prevents UV light penetration through the housing 306″ and minimizes exposure to the wearer.

The interior space of the housing 306″ is designed to promote ease of use and maximal UV (e.g. UVC) exposure to all parts of the mask sheet 310. In one embodiment, a cotton cloth was used as a sample mask sheet 310. As with the housing 306′, rods 330′ are used to define the track (e.g. serpentine path) that the mask sheet 310 follows when inside the housing 306″, creating a zig-zag pattern that prevents the material of the mask sheet 310 from folding in on itself and blocking areas from being exposed to UV light from the LED light strips 120′. Unlike the housing 306′ where glass rods 330 are used, in the housing 306″ polycarbonate was the selected material for these rods 330′ because it is permeable to UV light (e.g. UVC), allowing the germicidal radiation to reach the areas of the mask sheet 310 in direct contact with the rods 330′.

As further shown in FIG. 4C, the rods 330′ are rotatable within the housing 306″. In one embodiment, ball bearings 410 are provided and allow the rods 330′ to rotate while the mask sheet 310 glides along the arcuate path (e.g. serpentine path) within the housing 306″. The roller mechanism 314 is employed to retract and deploy the mask sheet 310. In an embodiment, one side of the roller mechanism 314 contains an anchor which attaches to the left end cap 340a, and the other side of the roller mechanism 314 contains the ratchet mechanism (e.g. ratchet gear 114) which slips when the mask sheet 310 is being extracted. As the mask sheet 310 is pulled from the housing 306″ (from the retracted position 102 to the extended position 103), two straps 360a, 360b (FIGS. 3N and 3O) attached to the bottom of the mask sheet 310 cause the roller mechanism 314 to rotate, adding tension to a spring inside the roller mechanism 314. Upon complete extraction to the extended position 103, the ratchet mechanism (e.g. ratchet gear 114) holds the spring tension until the retractor button 113 is pressed, causing the clutch to disengage, allowing the spring tension to be released, which pulls the mask sheet 310 back to the retracted position 102. In an embodiment, the roller mechanism 314 includes two guides surrounding the roller mechanism which act as a spool for the incoming straps 360a, 360b.

In an embodiment, a plurality of LED strips 120′ are provided, where each LED strip includes a plurality of LEDs. In one embodiment, four LED strips 120a′ through 120d′ are provided within the housing 306″. In an example embodiment, one LED strip 120a′ is mounted within a top of the interior space of the housing 306″, two LED strips 120b′, 120c′ are mounted within a front of the interior space of the housing 306″ and one LED strip 120d′ is mounted within a back of the interior space of the housing 306″. In this example embodiment, the front of the interior space of the housing 306″ faces the outer surface of the mask sheet 310 in the retracted position 102 (e.g. that is oriented at the surroundings of the wearer in the extended position 103) and the back of the interior space of the housing 306″ faces the inner surface of the mask sheet 310 in the retracted position 102 (e.g. that is oriented at the wearer in the extended position 103). In an example embodiment, each strip 120′ has seven 270 nm UVC-producing LEDs (28 lamps total) (e.g., cleanUV™, Waveform Lighting, Vancouver, Wash.). In these embodiments, the plurality of LED strips 120′ are placed in different locations around the interior space of the housing 306″ to provide exposure to all surfaces of the mask sheet 310 (see FIG. 4D). In some embodiments, the positioning of the LED strips 120′ can be selected and/or rearranged based on the light intensity incident on the mask sheet 310 within the housing (e.g. from the method 850).

In some embodiments, lithium ion batteries 312′ are provided as an internal power source. In this embodiment, two rechargeable 3.7V lithium-ion batteries (e.g., lithium-ion cylindrical battery, Adafruit, New York City, N.Y.) serve as the power source for the apparatus 400. In one embodiment, the LEDs 120′ require a 12V power source, and therefore two isolated step-up voltage regulators 342′ (e.g. step-up regulator, Pololu Robotics and Electronics, Las Vegas, Nev.) were used to generate a 12V output. In one example embodiment, the batteries 312′ are confined in a separate space from the mask sheet 310 in the housing 306″ and can easily be accessed and removed for recharging with the removal of the rear battery cover 348. In some embodiments, a push-button switch (not shown) can be used (e.g., Push-button switch 1A, CW Industries, Southampton, Pa.) and allows for the LED strips 120′ to be turned on and off. Additionally, in these embodiments, a micro-USB port 322 (FIG. 3H) is provided for recharging the batteries to provide increased ease of use (e.g., micro-LiPo charger with micro-USB jack, Adafruit, New York City, N.Y.). In one example embodiment, a total combined weight of the apparatus 400 including the rechargeable batteries 312 is about 242.9 grams.

FIG. 3K is a block diagram that illustrates an example of the apparatus 300 of FIGS. 3B through 3H, the apparatus 300′ of FIGS. 3I through 3J or the apparatus 400 of FIGS. 4A through 4D, according to an embodiment. In an embodiment, the block diagram of FIG. 3K is similar to the block diagram of FIG. 2D with the exception that multiple UV LED panels 120a through 120e (or LED strips 120a′ through 120d′) are mounted within the interior space of the housing 306, 306′, 306″ and communicatively coupled with the controller 101. Thus, the controller 101 transmits a first signal to the UV LED panels 120a through 120e (or LED strips 120a′ through 120d′) to initiate the sterilization process and a second signal to the UV LED panels 120a through 120e (or LED strips 120a′ through 120d′) (e.g., after the threshold time period for sterilization has elapsed) to end the sterilization process. In another embodiment, the apparatus 300 of FIG. 3K includes the rolling mechanism 314 that is similar to the inner rod 130.

FIGS. 3L through 3M are images that illustrate an example of the rolling mechanism 314 of the apparatus 300 of FIGS. 3B through 3H, the apparatus 300′ of FIGS. 3I through 3J and the apparatus 400 of FIGS. 4A through 4D, according to an embodiment. FIGS. 3N through 3Q are images that illustrate an example of the mask sheet 310 of the apparatus 300′ of FIGS. 3I through 3J or apparatus 400 of FIGS. 4A through 4D in the extended position 103 such that the straps 360a, 360b are provided along the arcuate path within the housing 306, 306″. When the mask sheet 310 is moved from the extended position 103 (FIG. 3Q) to the retracted position 102 (FIG. 3P), the straps 360a, 360b are wrapped around the rolling mechanism 314 (e.g. inner rod) and the mask sheet 310 is moved into the interior space of the housing 306, 306′, 306″ such that the mask sheet 310 is provided along the arcuate path within the housing.

FIG. 8A is a flow chart that illustrates an example method 800 for providing a self-sterilizing sanitary barrier. Although steps are depicted in the flowchart of FIG. 8A and subsequent flowchart in FIG. 8B as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways.

In step 801, the apparatus 100, 300, 300′, 400 is positioned on the user. In one embodiment, in step 801 the neck support 104 of the apparatus 100, 300, 300′, 400 engages the user to support the housing 106, 306, 306′, 306″ adjacent the user (e.g., adjacent a base of the neck). In an example embodiment, in step 801 the neck support 104 is a necklace that is wrapped around the neck of the user and/or connected behind the neck of the user. In another example embodiment, in step 801 the neck support 104 includes a pair of members which engage opposite sides of the neck and/or shoulders, to secure the housing 106, 306, 306′, 306″ to the user. Although a neck support is discussed in this embodiment of step 801, in other embodiments any means can be used to support the apparatus on the user (e.g., such that the housing is positioned near a base of the neck and below the chin).

In step 802, the mask sheet 110, 310 is donned on the user. In one embodiment, in step 802 the mask sheet 110, 310 is moved from the retracted position 102 to the extended position 103 to cover the portion of the face of the user. In an example embodiment, in step 802 the user holds the ear loops 108a, 108b and pulls them up and hooks them around their ears. In this example embodiment, pulling the ear loops 108a, 108b upward causes the mask sheet 110, 310 to move from the retracted position 102 to the extended position 103 (e.g., via rotation of the rolling mechanism 314 and/or the inner loop 130 and ratchet gear 114). In an example embodiment, different size mask sheets 110, 310 are provided for different users (e.g., child, adult, etc.). In other embodiments, step 802 is performed using features other than ear loops to don the mask sheet (e.g., a loop that extends around a back of the head, an adhesive that secures the mask sheet to the face of the user without ear loops, etc.). In still other embodiment, in step 802 the user opens the cover 406 and grasps the ear loops 108a, 108b positioned at the ear strap holder 404 and pulls the mask sheet 310 out of the housing 306″ by moving the ear loops 108a, 108b.

In step 804, the mask sheet 110, 310 is doffed from the user. In an embodiment, in step 804 the user no longer needs to wear the mask sheet 110, 310 in the extended position 103 (e.g., is home alone) and thus wants to move the mask sheet 110, 310 to the retracted position 102. In an example embodiment, in step 804 the user engages the switch 113 which causes the mask sheet 110, 310 to move from the extended position 103 to the retracted position 102 (e.g., by causing the pawl 112 to disengage the ratchet gear 114 and thus permit the ratchet gear 114 to rotate the inner loop 130 or rolling mechanism 314 within the housing 106, 306, 306′, 306″). In an example embodiment, in step 804 the mask sheet 310 is moved to the retracted position 102 and into the interior space of the housing 306, 306′, 306″ and along the serpentine path (e.g., into the slot 311 between the layers 308a, 308b in the housing 306 or around the rollers 330a through 330c in the housing 306′ or around rollers 330′ in the housing 306″).

In step 806, radiation is emitted from the light source within the housing 106 to sterilize the mask sheet 110, 310 in the retracted position 102. In an embodiment, in step 806 the sensor 107 detects that the mask sheet 110, 310 has moved to the retracted position 102 and transmits the signal to the controller 101 which subsequently transmits a signal to the UV LED panel 120 (or LED strips 120′) to cause UV light 121 to be emitted (e.g., for the minimum time period) and sterilize the mask sheet 110 in the retracted position 102 within the housing 106. In one embodiment, in step 806 upon receiving the signal from the sensor 107, the controller 101 transmits the signal to each of the UV LED panels 120a through 120e (or LED strips 120′) to cause UV light 121 to be emitted (e.g., for the minimum time period) and sterilize the mask sheet 310 provided along the serpentine path within the housing 306, 306′, 306″. In some embodiments, as the radiation from the light source is sterilizing the mask sheet 110, 310, the mask sheet 110, 310 cannot be moved from the retracted position 102 to the extended position 103. This advantageously provides a safety feature so that the mask sheet 110, 310 is not used in the extended position 103 until it is sterilized in step 806. In another embodiment, in step 806 the user can optionally input data to the controller 101 to vary one or more parameters of the sterilization of the mask sheet. In one embodiment, the user can input data using an input device (e.g., keypad 1107 or touch screen display 1107 of the mobile terminal 1101) which is in wireless communication with the sensor 107 and/or the UV LED panels 120. In this example embodiment, upon receiving first data (e.g., an exposure time, a UV intensity, etc.) the controller 101 uses equation 1 to determine second data (e.g. the exposure time, the UV intensity, etc.) in order to sterilize the mask sheet. In an example embodiment, where a user inputs a desired exposure time in step 806, the controller 101 uses equation 1 to calculate the minimum UV intensity to achieve a threshold UV dose (stored in memory) for the mask sheet. In another example embodiment, where the user inputs a desired UV intensity in step 806, the controller 101 uses equation 1 to calculate the exposure time to achieve the threshold UV dose (stored in memory). In other embodiments, method 850 is performed in order to estimate the value of the UV intensity in equation 1 and the controller 101 then uses this value of the UV intensity and a known value of the UV dose to compute the exposure time. In this embodiment, the computed exposure time is then used as the minimum time period during which UV light 121 is emitted.

In an embodiment, in step 806 the radiation emitted from the light source is configured to apply a radiation dosage to the back and front sides of the mask sheet 310. In an example embodiment, the radiation dosage is selected to inactivate a maximum amount of pathogens residing on the back and front surfaces of the mask sheet. In some embodiments, the radiation dosage applied to the back side of the mask sheet 310 is different from the radiation dosage applied to the front side of the mask sheet 310. For purposes of this description, the “back” of the mask sheet is defined as the side of the mask sheet facing the user when wearing the mask sheet and the “front” of the mask sheet is defined as the side of the mask sheet oriented away from the user (e.g. to the surroundings of the user). FIGS. 3R and 3S are images that illustrate an example of gradient figures 370, 380 of UV energy dosage applied to the respective back and front of the mask sheet, according to an embodiment. In an embodiment, the gradient figure 370 depicts one example of UV energy dosage applied to the back side of the mask sheet 310 during step 806 and the gradient figure 380 depicts one example of UV energy dosage applied to the front side of the mask sheet 310 during step 806. The horizontal axis 374 of both FIGS. 370, 380 indicates a horizontal dimension of the mask sheet 310 in arbitrary units (e.g. mm) and the vertical axis 376 of both FIGS. 370, 380 indicates a vertical dimension of the mask sheet 310 in arbitrary units (e.g. mm). The color scale 372 indicates a color associated with each value of UV intensity applied to the mask sheet 310 during step 806. As shown in FIGS. 3R and 3S, in one embodiment the UV energy dosage is higher on the front side of the mask sheet 310, since the front side of the mask sheet 310 is more susceptible to collecting pathogens (e.g. user touching the outside of the mask sheet, pathogens from the surroundings of the user contacting the outside of the mask sheet, etc.). The gradient FIGS. 370, 380 are merely one example embodiment of UV energy dosage applied to the back and front of the mask sheet 310 during step 806. In other embodiments, the UV energy dosage can be applied to the back and front of the mask sheet 310 which differs from the gradient FIGS. 370, 380. The method 850 of FIG. 8B provides more details on how the UV intensity values of the gradient FIGS. 370, 380 is determined.

In step 808, after the mask sheet 110, 310 is sterilized in step 806 the mask sheet 110, 310 is donned by the user by moving the mask sheet 110, 310 from the retracted position 102 to the extended position 103.

In order to sterilize the mask sheet, the exposure time of equation 1 (or minimum time period) needs to be determined during which the mask sheet is radiated with light from the light source. The mask sheet 310 is radiated with UV light 121 from the UV light source (e.g. LED strips 120′) during step 806 and thus the value of the exposure time needs to be determined. The inventors of the present invention recognized that in order to estimate the exposure time in equation 1, an estimate of the UV intensity of UV light 121 incident on the mask sheet 310 within the housing 306, 306′, 306″ needs to be determined. In one embodiment, the embodiments disclosed herein disclose a method for estimating a value of the UV intensity at a plurality of locations along the front side and back side of the mask sheet 310 withing the housing. In this embodiment, the estimate of the UV intensity in equation 1 is then obtained based on these estimated values of the UV intensity at the locations on the front and back side of the mask sheet 310 within the housing. In one example embodiment, the estimate of the UV intensity in equation 1 is obtained by using a minimum value among all of the estimated UV intensity values at all locations on the front and back side of the mask sheet 310 within the housing. Method 850 of FIG. 8B will now be discussed, which teaches one example of a method for estimating these UV intensity values at a plurality of locations along the back and front side of the mask sheet within the housing.

FIG. 8B is a flow chart that illustrates an example method 850 for calibrating the apparatus of FIG. 4A, according to an embodiment. In one embodiment, in step 852 a plurality of sensors (e.g. UV light sensors) are positioned at multiple distance separation from the UV light source (e.g. LED strip 120′). In an example embodiment, the manufacturer of the UV light source provides data including a known intensity of the UV light at multiple distances separation from the UV light source. The first two columns of Table 1 below is an example of such data provided by the manufacturer.

TABLE 1
Given intensity and measured resistance of a single
LED lamp at set distances from the UV sensor.

| | Intensity | Measured resistance | |
|---|---|---|---|
| Distance (cm) | (μW/cm²) | (MΩ) | 1/Rₘ |
| 2.54 | 118.4 | 0.20 | 5.12 |
| 5.08 | 78.2 | 0.72 | 1.39 |
| 7.62 | 61.5 | 1.52 | 0.66 |
| 10.16 | 48.6 | 3.30 | 0.30 |

In this example embodiment, the multiple distances of separation from the light source include 1 inch (2.54 cm), 2 inches (5.08 cm), 3 inches (7.62 cm) and 4 inches (10.16 cm). However, the data provided by the manufacturer may include a different number or different distance values. In step 852 the plurality of sensors are positioned at the multiple distance separations from the UV light source that corresponds to the manufacturer.

In step 854, after positioning the plurality of sensors at the multiple distance separation from the UV light source, the light source (e.g. UV strip 120′) is activated to illuminate the plurality of sensors with light (e.g. UV light 121).

In step 856, after illuminating the plurality of sensors in 854 a value of an electrical parameter is measured for each sensor. In one embodiment, the electrical parameter is a resistance of each sensor being illuminated with the UV light from the UV light source. In one example embodiment, the third column of Table 1 above shows example resistance values measured for each sensor positioned at each respective distance separation from the light source. In another example embodiment, in step 856 a value of a second electrical parameter is calculated based on the value of the first electrical parameter measured. In an example embodiment, the second parameter is a conductance at each sensor, which is an inverse value of the measured resistance at each sensor. In this example embodiment, the fourth column of Table 1 above shows example conductance values for each sensor.

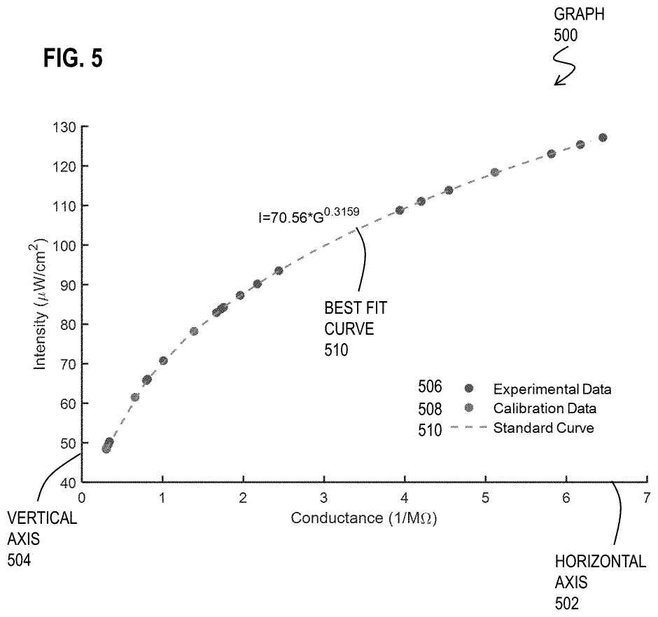

In step 858, data is plotted on a graph including the measured data from step 856 and the known intensity values of light from the light manufacturer. In one embodiment, a best fit curve is then calculated for the plotted data. In an example embodiment, FIG. 5 depicts a graph 500 with plotted data in step 858. The horizontal axis 502 is conductance in units of Ω⁻¹. The vertical axis 504 is light intensity in units of μW/cm². The calibration data 508 plotted in the graph 500 using the values in the fourth column of Table 1 along the horizontal axis 502 and the values in the second column of Table 1 along the vertical axis 504. A best fit curve 510 is then calculated through the calibration data 508 points (e.g. using a least-square fit). In an example embodiment, the best fit curve 510 is:

I=70.536 G^{0.3159}. (2)

where I is the intensity of the UV light incident on the sensor and G is the conductance at each sensor (based on the inverse of the measured resistance value). Equation 2 is merely one example of a best fit curve based on the specific data obtained for a particular LED source and particular sensors. Accordingly, equation 2 will be different depending on the type of light source (e.g. LED strip, etc.) and the type of sensors.

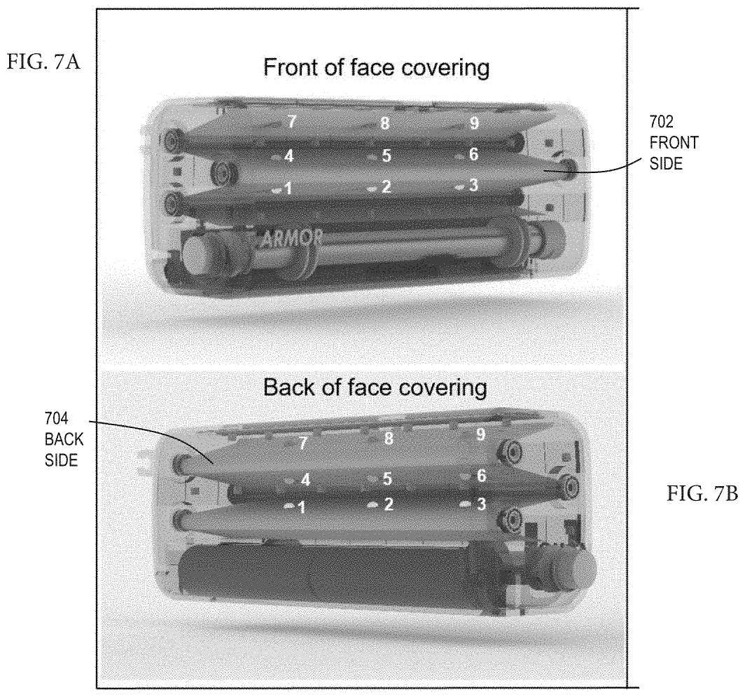

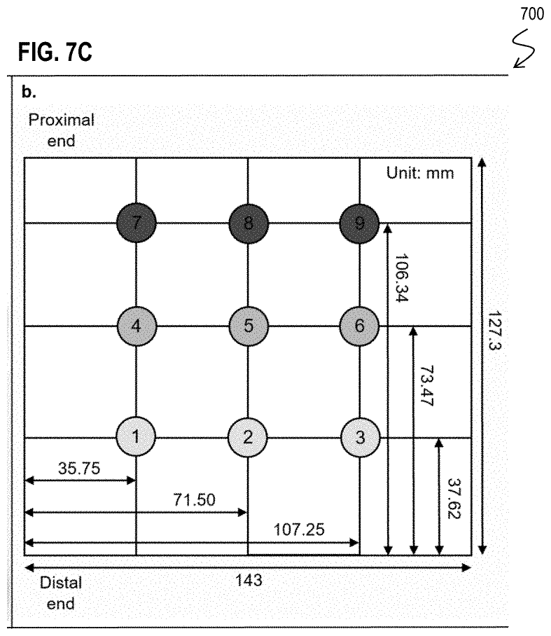

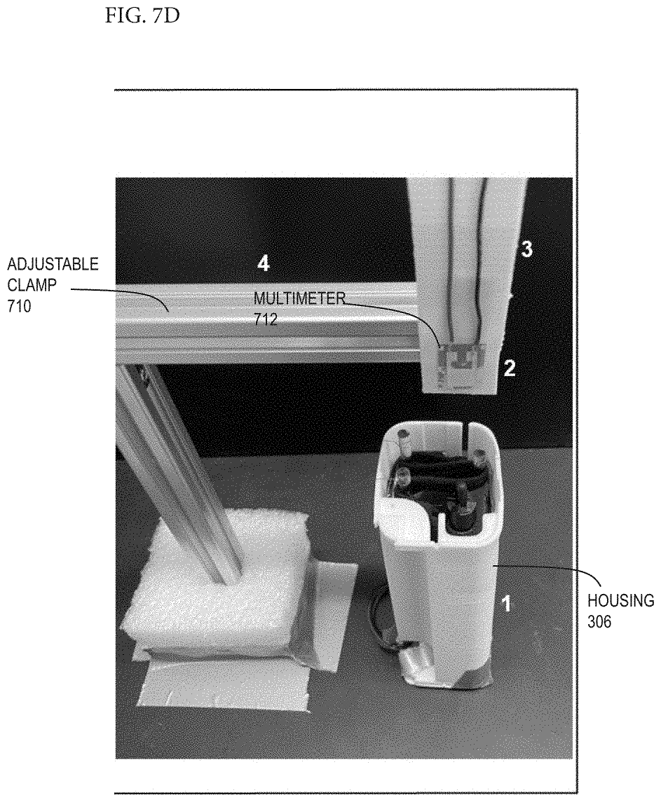

In steps 860 and 862, a plurality of sensors are positioned at a plurality of locations on the front and back side of the mask sheet 310 after which the mask sheet 310 is moved to the retracted position 102 within the housing 306″. The UV light intensity is estimated at each respective sensor location on the front and back side of the mask sheet 310. FIGS. 7A and 7B depict an image that shows a first set of sensors 1 through 9 positioned on the front side 702 of the mask sheet 310 and a second set of sensors 1 through 9 positioned on the back side 704 of the mask sheet 310. FIG. 7C depicts one example of a spatial arrangement of the sensors 1 through 9 on each side of the mask sheet 310. In some embodiments, step 860 is performed separately for each sensor (e.g. one sensor at a time is positioned on the front or back side of the mask sheet 310 and the UV intensity is estimated).

In an embodiment, step 860 is performed using the assembly shown in FIG. 7D. In an embodiment, the housing 306″ is provided with a mask sheet 310 (not shown) within the housing in the retracted position 102. A sensor (not shown) is positioned within the housing at one of the sensor locations 1 through 9 of the front or back side of the mask sheet 310. The light source (e.g. LED strips 120a′ through 120d′) is then illuminated and a resistance of the sensor is measured (e.g. using the multimeter 712). In one embodiment, the measured resistance is then recorded for that particular sensor location 1 through 9 and this measurement is repeated (e.g. eighteen times) for each sensor location 1 through 9 of the front and back side of the mask sheet 310. In one embodiment, an adjustable clamp 710 is provided that can be moved in one or more dimensions so that the sensor (not shown) can be correspondingly positioned at each of the sensor locations 1 through 9 of the front and back side of the mask sheet 310 and then resistance then measured. After recording these resistance values, the corresponding conductance values are measured (e.g. inverse of the resistance value). Using the best fit curve 510 of FIG. 5 (e.g. equation 2), the light intensity at each sensor is then calculated, based on the measured conductance value. This data including the calculated conductance value and estimated light intensity at each sensor location 1 through 9 is shown as experimental data 506 points on the graph 500 of FIG. 5.

In an embodiment, in step 864 the UV intensity of equation 1 is estimated, based on the data obtained in steps 860 and 862. In one embodiment, a lowest UV intensity value among all of the sensors 1 through 9 on the front and back side of the mask sheet 310 is used for the UV intensity in equation 1 (e.g. a lowest value among the eighteen intensity values obtained in step 862). The inventors recognized that this advantageously ensures that the mask sheet 310 will be sterilized within the calculated exposure time, since the minimum UV intensity value incident on the mask sheet 310 is used to compute the exposure time. In other embodiments, a different parameter (e.g. average, mean, percentile value, etc.) of the estimated UV intensity at each of the sensors 1 through 9 on the front and back side of the mask sheet 310 is used for the UV intensity in equation 1 when computing the exposure time. In an example embodiment, the computed exposure time using equation 1 is stored in the memory 105 of the controller 101 and is used to activate and deactivate the light sources (e.g. LED strips 120a′ through 120d′).

### 2. EXAMPLE EMBODIMENTS

In one embodiment, before UVC light intensity is estimated within the housing 306″ in step 862, standard resistance values of the sensors at set distances were recorded in step 856. The standard calibration curve (e.g. best fit curve 510 of FIG. 5) is then created.

In one embodiment, the sensor utilized in steps 856 and 860 are fabricated ZnO-based UV sensors that quantitively measure the level of UV intensity from the electrical resistance. In this embodiment, the level of resistance of the sensor is inversely proportional to the level of UV intensity. This resistance response curve was used to calculate UV light intensity at different areas on the mask sheet 310 in step 862. As expected in the calibration data, as sensor distance from the LED lamp increased, electrical resistance also increased and light intensity decreased (see Table 1 data above). The graph 500 of the standard curve 510 revealed a nonlinear relationship between intensity and conductance, and the equation of the best fit curve 510 was used to calculate light intensity in step 862 at different areas (e.g. sensor locations 1 through 9 on the front and back side) on the mask sheet 310 from the experimental resistance measurements obtained in step 856.

In one embodiment, Table 2 shows an example of measured data in step 860 and 862.

TABLE 2
Experimental resistance and calculated intensity values
at 18 different locations on the face covering.

| | | Experimental | | Calculated |
|---|---|---|---|---|
| | Sensor | resistance | | intensity |
| | position | (MΩ) | 1/Rₑ | (μW/cm²) |
| Front of face | 1 | 0.57 | 1.75 | 84.27 |
| covering | 2 | 0.58 | 1.72 | 83.81 |
| | 3 | 0.6 | 1.67 | 82.92 |
| | 4 | 0.162 | 6.17 | 125.40 |
| | 5 | 0.155 | 6.45 | 127.16 |
| | 6 | 0.22 | 4.55 | 113.84 |
| | 7 | 0.51 | 1.96 | 87.29 |
| | 8 | 0.46 | 2.17 | 90.18 |
| | 9 | 0.41 | 2.44 | 93.52 |
| Back of face | 1 | 1.25 | 0.80 | 65.76 |
| covering | 2 | 1.23 | 0.81 | 66.10 |
| | 3 | 0.99 | 1.01 | 70.79 |
| | 4 | 3.3 | 0.30 | 48.39 |
| | 5 | 2.92 | 0.34 | 50.30 |
| | 6 | 3.09 | 0.32 | 49.41 |
| | 7 | 0.172 | 5.81 | 123.05 |
| | 8 | 0.238 | 4.20 | 111.05 |
| | 9 | 0.254 | 3.94 | 108.79 |

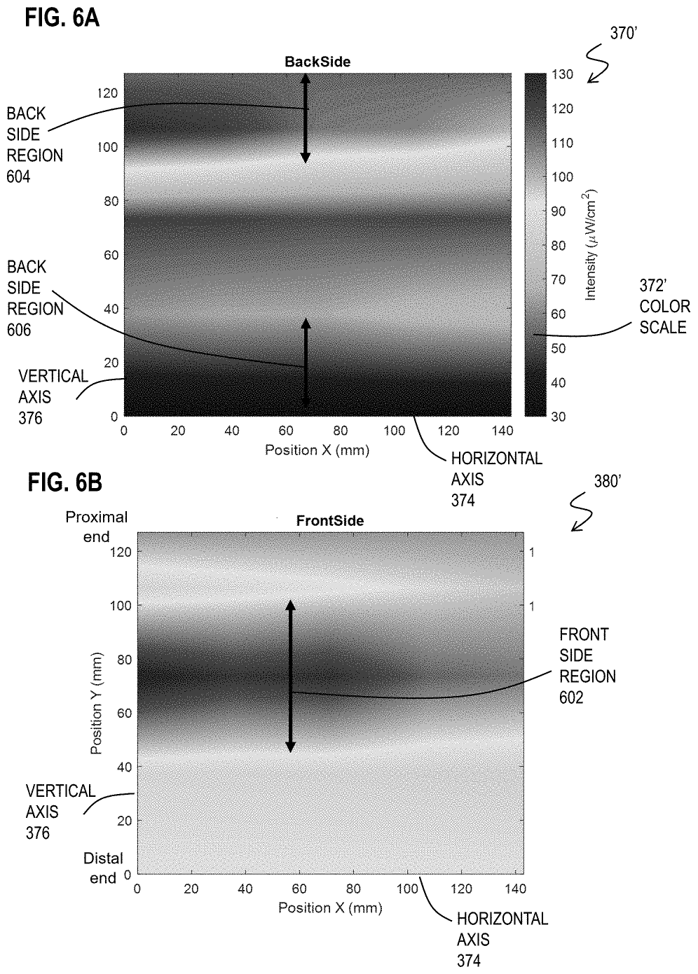

The front side 702 (FIG. 7A) of the mask sheet 310, designated as the side exposed to the wearer's environment, received UVC light at an intensity range from about 82.92 μW/cm² to about 127.16 μW/cm² (Table 2). FIG. 6B shows a gradient figure 380′ with estimated intensity values (using color scale 372′) on the front side 702 of the mask sheet 310. It was found that sensor positions 4, 5, and 6 (e.g. along front side region 602 in FIG. 6B) on the front side 702 received the highest intensity light, which means that these areas of the mask sheet 310 are most directly exposed to the LED lamps.

The back side 704 (FIG. 7B) of the mask sheet 310 is designated as the side in contact with the wearer's nose and mouth. After experimentation, it was found that the light intensity on this side ranged from about 48.39 μW/cm² to about 123.05 μW/cm² (Table 2). The similar upper range of intensity values from front to back sides 702, 704 suggests that there are areas on both surfaces (e.g. front side region 602 in FIG. 7B and back side region 604 in FIG. 6A) that receive direct exposure to the UVC-producing LED lights. Sensor locations 1, 2, and 3 on the back side 704 of the mask sheet 310 (e.g. corresponding to back side region 606 in FIG. 6A) of the mask sheet 310 received the lowest intensity UVC, which can be attributed to the distance from the LED lamps and the non-perpendicular angle to the incoming light the face covering rests at.

In some embodiments, it was not feasible to place the sensor in some areas inside the housing 306″ in step 860. To obtain the gradient FIGS. 370′ and 380′, linear forecasting was used to extrapolate the intensity data obtained in step 862 to the edges of the mask sheet 310. After calculation, the area of least UVC exposure was determined to be the bottom edge of the back side 704 (e.g. back side region 606 in FIG. 6A) of the mask sheet 310, with a light intensity value of about 29.06 μW/cm². Given the expected areas of greatest and least exposure in the design, all intensity values calculated in step 862 using the linear forecasting method were determined to be reasonable. In some embodiments, the number and/or arrangement of the LED strips 120′ can be varied, based on the data obtained in steps 862 and 864. For example, if the lowest value of the intensity obtained in step 864 is not sufficient to sterilize the mask sheet within a reasonable time, the placement and/or number of LED strips 120′ can be varied and then method 850 is repeated until a sufficient intensity value is obtained in step 864 which can sterilize the mask sheet within a reasonable time (e.g. less than 5 minutes).

In step 864, the exposure time required to inactivate different pathogens was calculated by dividing the given required energy dosage (e.g. predetermined based on the type of pathogen) by the lowest experimentally determined UVC light intensity value from the light intensity values calculated in step 862 (e.g. about 29.06 μW/cm2). Table 3 below shows required energy dosages for various types of pathogens.

TABLE 3
Required energy dosages for inactivation and calculated exposure
time values for seven common pathogens.^{15, 16, 17, 18}

| Pathogen | Required energy | Exposure time |
|---|---|---|
| (% inactivation) | dosage (mJ/cm²) | (s) |
| SARS-CoV-2 (99) | 5.0 | 182.87 |
| HCoV-229E (99.9) | 1.7 | 71.14 |
| HCoV-OC43 (99.9) | 1.2 | 50.22 |
| Mycobacterium tuberculosis (99.9) | 3.3 | 113.54 |
| H1N1 Influenza (99.9) | 3.8 | 130.75 |
| Escherichia coli (99.9) | 2.4 | 82.58 |
| Streptococcus pyogenes (99.9) | 1.2 | 41.29 |

Pathogens that are more susceptible to UVC radiation have a lower required energy dosage, and therefore take a shorter amount of time to become inactivated. Streptococcus pyogenes, the bacteria that causes the common illness “strep throat,” undergoes a 3-log reduction in under 42 seconds of exposure to the LED lamps inside the housing. A more UVC-resilient pathogen such as SARS-CoV-2 requires a longer exposure time to become inactivated, approximately 183 seconds to reach a 2-log reduction.

Analysis of the experimental UVC intensity data revealed areas of the mask sheet 310 with high exposure, and areas with less exposure. The contour plots of intensity reveal that the locations with the highest UV exposure are the center (y=40-100 mm) and top (y=110-127 mm) of the front side 702 (FIG. 6B), and the top (y=95-127 mm) of the back side 704 of the face covering (FIG. 6A).

In one embodiment, the apparatus disclosed herein was designed to be an effective and elegant solution for minimizing mask sheet 310 contamination and disposable mask waste. Using existing knowledge on the ability of ultraviolet C radiation to inactivate SARS-CoV-2 and other pathogens, the effectiveness of the apparatus in sanitizing a reusable mask sheet could be tested indirectly. The front side 702 of the mask sheet 310 faces away from the wearer and is exposed the most to the environment. Therefore, it is advantageous that this side receives the highest intensity of the germicidal UVC light because it is likely the most easily contaminated. The back side 704 of the mask sheet 310 faces the wearer, and it is important that this surface receives sufficient exposure during sanitization as well. The upper half of the back side 704 (e.g. back side region 604 in FIG. 6A) is in contact with the nose and mouth of the user, so it is significant that this area receives a UV intensity greater than 70 μW/cm2. The bottom of the back side 704 (e.g. back side region 606 in FIG. 6A) of the mask sheet 310 is in contact with the wearer's chin and neck area, which means that it likely receives the least amount of contamination. As a result, it is reasonable that this area receives a lesser intensity of the germicidal radiation.

In some embodiments, regardless of the intensity in each location on the mask sheet 310, all areas on both sides 702, 704 are exposed to the UVC light. This means that even the areas of least intensity can reach the required energy dosage to be sanitized of different pathogens if the exposure time is sufficient. In an example embodiment, in step 864 the exposure time is calculated by dividing the required energy dosage (e.g. from Table 3) by the light intensity (e.g. from step 864). In some embodiments, this number was then recalculated for specific pathogens by dividing by the percentage of light energy produced by the 270 nm LED lamps used in the housing compared to the 254 nm light used to study SARS-CoV-2 and the 222 nm light used to study HCoV-229E and HCoV-OC43. [15, 16]. Use of UVC radiation at 270 nm is a proven bactericidal and virucidal method.[14] In this example embodiment, the recalculated exposure times for HCoV-229E, HCoV-OC43, and SARS-CoV-2 therefore reflect the worst-case ability for the apparatus to inactivate more than 99.9% of these viruses. Some viruses such as SARS-CoV-2 and H1N1 Influenza require a higher UVC energy dosage to reach the same level of sterilization. As a result, the exposure time can be extended accordingly to achieve broad-spectrum sterilization. The longest exposure time was determined to be at the bottom of the back side 704 (e.g. back side region 606 in FIG. 6A) of the mask sheet 310, taking 183 seconds for SARS-CoV-2, the pathogen with the highest UVC resilience of those tested, to be 99% inactivated. Therefore, in this embodiment, the 183 seconds is also the time that the apparatus takes to sanitize all areas of the mask of all of the tested pathogens.

In one embodiment, the high observed light intensity on both sides 702, 704 of the mask sheet 310 and the relatively short time required to disinfect when compared to traditional washing methods indicates that the apparatus disclosed herein is successful in achieving the objective of sterilizing the mask sheet 310 with enhanced efficiency. The apparatus disclosed herein presents benefits to front-line healthcare workers by eliminating pathogens present on their mask sheets, therefore reducing the spread of deadly infectious diseases. In between visiting patients, the mask sheet 310 can be removed and sanitized in just a couple minutes, reducing the accumulation of bacteria and viruses on the mask sheet 310. Use of apparatus disclosed herein instead of traditional disposable masks can also significantly reduce the amount of medical waste that ends up in landfills.

In some embodiments, both the size and weight of the apparatus can be reduced with additional optimization. Given the short amount of time currently required to sanitize the mask sheet 310, the number of LED lamps can be reduced, eliminating the need for two batteries, and reducing the size and weight of apparatus while still keeping the exposure time to a few minutes. Additionally, a cotton mask sheet was used in this version, which can allow infected water droplets to settle into the material and also acts as a poor filter for microbes. This could result in reduced exposure of pathogens to the UVC and decreased germicidal effects on the mask sheet. As a solution, in other embodiments nanofiber is used as an alternative to cotton. In this embodiment, nanofiber is used as a common mask filter material, has a high filtration efficiency, and is UV resilient over long periods of time, making it the ideal material for use in the apparatus [19, 20]. In some embodiments, the design of the apparatus is effective at providing a lethal dose of ultraviolet-C radiation to the pathogens on the surface of a mask sheet, therefore protecting the wearer from infection and reducing unnecessary waste, while still leaving room for additional design improvement.

### 3. HARDWARE OVERVIEW

FIG. 9 is a block diagram that illustrates a computer system 900 upon which an embodiment of the invention may be implemented. Computer system 900 includes a communication mechanism such as a bus 910 for passing information between other internal and external components of the computer system 900. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit)). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range. Computer system 900, or a portion thereof, constitutes a means for performing one or more steps of one or more methods described herein.

A sequence of binary digits constitutes digital data that is used to represent a number or code for a character. A bus 910 includes many parallel conductors of information so that information is transferred quickly among devices coupled to the bus 910. One or more processors 902 for processing information are coupled with the bus 910. A processor 902 performs a set of operations on information. The set of operations include bringing information in from the bus 910 and placing information on the bus 910. The set of operations also typically include comparing two or more units of information, shifting positions of units of information, and combining two or more units of information, such as by addition or multiplication. A sequence of operations to be executed by the processor 902 constitutes computer instructions.

Computer system 900 also includes a memory 904 coupled to bus 910. The memory 904, such as a random access memory (RAM) or other dynamic storage device, stores information including computer instructions. Dynamic memory allows information stored therein to be changed by the computer system 900. RAM allows a unit of information stored at a location called a memory address to be stored and retrieved independently of information at neighboring addresses. The memory 904 is also used by the processor 902 to store temporary values during execution of computer instructions. The computer system 900 also includes a read only memory (ROM) 906 or other static storage device coupled to the bus 910 for storing static information, including instructions, that is not changed by the computer system 900. Also coupled to bus 910 is a non-volatile (persistent) storage device 908, such as a magnetic disk or optical disk, for storing information, including instructions, that persists even when the computer system 900 is turned off or otherwise loses power.

Information, including instructions, is provided to the bus 910 for use by the processor from an external input device 912, such as a keyboard containing alphanumeric keys operated by a human user, or a sensor. A sensor detects conditions in its vicinity and transforms those detections into signals compatible with the signals used to represent information in computer system 900. Other external devices coupled to bus 910, used primarily for interacting with humans, include a display device 914, such as a cathode ray tube (CRT) or a liquid crystal display (LCD), for presenting images, and a pointing device 916, such as a mouse or a trackball or cursor direction keys, for controlling a position of a small cursor image presented on the display 914 and issuing commands associated with graphical elements presented on the display 914.

In the illustrated embodiment, special purpose hardware, such as an application specific integrated circuit (IC) 920, is coupled to bus 910. The special purpose hardware is configured to perform operations not performed by processor 902 quickly enough for special purposes. Examples of application specific ICs include graphics accelerator cards for generating images for display 914, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 900 also includes one or more instances of a communications interface 970 coupled to bus 910. Communication interface 970 provides a two-way communication coupling to a variety of external devices that operate with their own processors, such as printers, scanners and external disks. In general, the coupling is with a network link 978 that is connected to a local network 980 to which a variety of external devices with their own processors are connected. For example, communication interface 970 may be a parallel port or a serial port or a universal serial bus (USB) port on a personal computer. In some embodiments, communications interface 970 is an integrated services digital network (ISDN) card or a digital subscriber line (DSL) card or a telephone modem that provides an information communication connection to a corresponding type of telephone line. In some embodiments, a communication interface 970 is a cable modem that converts signals on bus 910 into signals for a communication connection over a coaxial cable or into optical signals for a communication connection over a fiber optic cable. As another example, communications interface 970 may be a local area network (LAN) card to provide a data communication connection to a compatible LAN, such as Ethernet. Wireless links may also be implemented. Carrier waves, such as acoustic waves and electromagnetic waves, including radio, optical and infrared waves travel through space without wires or cables. Signals include man-made variations in amplitude, frequency, phase, polarization or other physical properties of carrier waves. For wireless links, the communications interface 970 sends and receives electrical, acoustic or electromagnetic signals, including infrared and optical signals, that carry information streams, such as digital data.

The term computer-readable medium is used herein to refer to any medium that participates in providing information to processor 902, including instructions for execution. Such a medium may take many forms, including, but not limited to, non-volatile media, volatile media and transmission media. Non-volatile media include, for example, optical or magnetic disks, such as storage device 908. Volatile media include, for example, dynamic memory 904. Transmission media include, for example, coaxial cables, copper wire, fiber optic cables, and waves that travel through space without wires or cables, such as acoustic waves and electromagnetic waves, including radio, optical and infrared waves. The term computer-readable storage medium is used herein to refer to any medium that participates in providing information to processor 902, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read. The term non-transitory computer-readable storage medium is used herein to refer to any medium that participates in providing information to processor 902, except for carrier waves and other signals.

Logic encoded in one or more tangible media includes one or both of processor instructions on a computer-readable storage media and special purpose hardware, such as ASIC *920.

Network link 978 typically provides information communication through one or more networks to other devices that use or process the information. For example, network link 978 may provide a connection through local network 980 to a host computer 982 or to equipment 984 operated by an Internet Service Provider (ISP). ISP equipment 984 in turn provides data communication services through the public, world-wide packet-switching communication network of networks now commonly referred to as the Internet 990. A computer called a server 992 connected to the Internet provides a service in response to information received over the Internet. For example, server 992 provides information representing video data for presentation at display 914.

The invention is related to the use of computer system 900 for implementing the techniques described herein. According to one embodiment of the invention, those techniques are performed by computer system 900 in response to processor 902 executing one or more sequences of one or more instructions contained in memory 904. Such instructions, also called software and program code, may be read into memory 904 from another computer-readable medium such as storage device 908. Execution of the sequences of instructions contained in memory 904 causes processor 902 to perform the method steps described herein. In alternative embodiments, hardware, such as application specific integrated circuit 920, may be used in place of or in combination with software to implement the invention. Thus, embodiments of the invention are not limited to any specific combination of hardware and software.

The signals transmitted over network link 978 and other networks through communications interface 970, carry information to and from computer system 900. Computer system 900 can send and receive information, including program code, through the networks 980, 990 among others, through network link 978 and communications interface 970. In an example using the Internet 990, a server 992 transmits program code for a particular application, requested by a message sent from computer 900, through Internet 990, ISP equipment 984, local network 980 and communications interface 970. The received code may be executed by processor 902 as it is received or may be stored in storage device 908 or other non-volatile storage for later execution, or both. In this manner, computer system 900 may obtain application program code in the form of a signal on a carrier wave.

Various forms of computer readable media may be involved in carrying one or more sequence of instructions or data or both to processor 902 for execution. For example, instructions and data may initially be carried on a magnetic disk of a remote computer such as host 982. The remote computer loads the instructions and data into its dynamic memory and sends the instructions and data over a telephone line using a modem. A modem local to the computer system 900 receives the instructions and data on a telephone line and uses an infra-red transmitter to convert the instructions and data to a signal on an infra-red a carrier wave serving as the network link 978. An infrared detector serving as communications interface 970 receives the instructions and data carried in the infrared signal and places information representing the instructions and data onto bus 910. Bus 910 carries the information to memory 904 from which processor 902 retrieves and executes the instructions using some of the data sent with the instructions. The instructions and data received in memory 904 may optionally be stored on storage device 908, either before or after execution by the processor 902.

FIG. 10 illustrates a chip set 1000 upon which an embodiment of the invention may be implemented. Chip set 1000 is programmed to perform one or more steps of a method described herein and includes, for instance, the processor and memory components described with respect to FIG. *9 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip. Chip set 1000, or a portion thereof, constitutes a means for performing one or more steps of a method described herein.

In one embodiment, the chip set 1000 includes a communication mechanism such as a bus 1001 for passing information among the components of the chip set 1000. A processor 1003 has connectivity to the bus 1001 to execute instructions and process information stored in, for example, a memory 1005. The processor 1003 may include one or more processing cores with each core configured to perform independently. A multi-core processor enables multiprocessing within a single physical package. Examples of a multi-core processor include two, four, eight, or greater numbers of processing cores. Alternatively, or in addition, the processor 1003 may include one or more microprocessors configured in tandem via the bus 1001 to enable independent execution of instructions, pipelining, and multithreading. The processor 1003 may also be accompanied with one or more specialized components to perform certain processing functions and tasks such as one or more digital signal processors (DSP) 1007, or one or more application-specific integrated circuits (ASIC) 1009. A DSP 1007 typically is configured to process real-world signals (e.g., sound) in real time independently of the processor 1003. Similarly, an ASIC 1009 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 1003 and accompanying components have connectivity to the memory 1005 via the bus 1001. The memory 1005 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 1005 also stores the data associated with or generated by the execution of one or more steps of the methods described herein.

FIG. 11 is a diagram of exemplary components of a mobile terminal 1101 (e.g., cell phone handset) for communications, which is capable of operating in the system of FIG. 2B, according to one embodiment. In some embodiments, mobile terminal 1101, or a portion thereof, constitutes a means for performing one or more steps described herein. Generally, a radio receiver is often defined in terms of front-end and back-end characteristics. The front-end of the receiver encompasses all of the Radio Frequency (RF) circuitry whereas the back-end encompasses all of the base-band processing circuitry. As used in this application, the term “circuitry” refers to both: (1) hardware-only implementations (such as implementations in only analog and/or digital circuitry), and (2) to combinations of circuitry and software (and/or firmware) (such as, if applicable to the particular context, to a combination of processor(s), including digital signal processor(s), software, and memory(ies) that work together to cause an apparatus, such as a mobile phone or server, to perform various functions). This definition of “circuitry” applies to all uses of this term in this application, including in any claims. As a further example, as used in this application and if applicable to the particular context, the term “circuitry” would also cover an implementation of merely a processor (or multiple processors) and its (or their) accompanying software/or firmware. The term “circuitry” would also cover if applicable to the particular context, for example, a baseband integrated circuit or applications processor integrated circuit in a mobile phone or a similar integrated circuit in a cellular network device or other network devices.

Pertinent internal components of the telephone include a Main Control Unit (MCU) 1103, a Digital Signal Processor (DSP) 1105, and a receiver/transmitter unit including a microphone gain control unit and a speaker gain control unit. A main display unit 1107 provides a display to the user in support of various applications and mobile terminal functions that perform or support the steps as described herein. The display 1107 includes display circuitry configured to display at least a portion of a user interface of the mobile terminal (e.g., mobile telephone). Additionally, the display 1107 and display circuitry are configured to facilitate user control of at least some functions of the mobile terminal. An audio function circuitry 1109 includes a microphone 1111 and microphone amplifier that amplifies the speech signal output from the microphone 1111. The amplified speech signal output from the microphone 1111 is fed to a coder/decoder (CODEC) 1113.

A radio section 1115 amplifies power and converts frequency in order to communicate with a base station, which is included in a mobile communication system, via antenna 1117. The power amplifier (PA) 1119 and the transmitter/modulation circuitry are operationally responsive to the MCU 1103, with an output from the PA 1119 coupled to the duplexer 1121 or circulator or antenna switch, as known in the art. The PA 1119 also couples to a battery interface and power control unit 1120.

In use, a user of mobile terminal 1101 speaks into the microphone 1111 and his or her voice along with any detected background noise is converted into an analog voltage. The analog voltage is then converted into a digital signal through the Analog to Digital Converter (ADC) 1123. The control unit 1103 routes the digital signal into the DSP 1105 for processing therein, such as speech encoding, channel encoding, encrypting, and interleaving. In one embodiment, the processed voice signals are encoded, by units not separately shown, using a cellular transmission protocol such as enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., microwave access (WiMAX), Long Term Evolution (LTE) networks, code division multiple access (CDMA), wideband code division multiple access (WCDMA), wireless fidelity (WiFi), satellite, and the like, or any combination thereof.

The encoded signals are then routed to an equalizer 1125 for compensation of any frequency-dependent impairments that occur during transmission though the air such as phase and amplitude distortion. After equalizing the bit stream, the modulator 1127 combines the signal with a RF signal generated in the RF interface 1129. The modulator 1127 generates a sine wave by way of frequency or phase modulation. In order to prepare the signal for transmission, an up-converter 1131 combines the sine wave output from the modulator 1127 with another sine wave generated by a synthesizer 1133 to achieve the desired frequency of transmission. The signal is then sent through a PA 1119 to increase the signal to an appropriate power level. In practical systems, the PA 1119 acts as a variable gain amplifier whose gain is controlled by the DSP 1105 from information received from a network base station. The signal is then filtered within the duplexer 1121 and optionally sent to an antenna coupler 1135 to match impedances to provide maximum power transfer. Finally, the signal is transmitted via antenna 1117 to a local base station. An automatic gain control (AGC) can be supplied to control the gain of the final stages of the receiver. The signals may be forwarded from there to a remote telephone which may be another cellular telephone, any other mobile phone or a land-line connected to a Public Switched Telephone Network (PSTN), or other telephony networks.

Voice signals transmitted to the mobile terminal 1101 are received via antenna 1117 and immediately amplified by a low noise amplifier (LNA) 1137. A down-converter 1139 lowers the carrier frequency while the demodulator 1141 strips away the RF leaving only a digital bit stream. The signal then goes through the equalizer 1125 and is processed by the DSP 1105. A Digital to Analog Converter (DAC) 1143 converts the signal and the resulting output is transmitted to the user through the speaker 1145, all under control of a Main Control Unit (MCU) 1103 which can be implemented as a Central Processing Unit (CPU) (not shown).

The MCU 1103 receives various signals including input signals from the keyboard 1147. The keyboard 1147 and/or the MCU 1103 in combination with other user input components (e.g., the microphone 1111) comprise a user interface circuitry for managing user input. The MCU 1103 runs a user interface software to facilitate user control of at least some functions of the mobile terminal 1101 as described herein. The MCU 1103 also delivers a display command and a switch command to the display 1107 and to the speech output switching controller, respectively. Further, the MCU 1103 exchanges information with the DSP 1105 and can access an optionally incorporated SIM card 1149 and a memory 1151. In addition, the MCU 1103 executes various control functions required of the terminal. The DSP 1105 may, depending upon the implementation, perform any of a variety of conventional digital processing functions on the voice signals. Additionally, DSP 1105 determines the background noise level of the local environment from the signals detected by microphone 1111 and sets the gain of microphone 1111 to a level selected to compensate for the natural tendency of the user of the mobile terminal 1101.

The CODEC 1113 includes the ADC 1123 and DAC 1143. The memory 1151 stores various data including call incoming tone data and is capable of storing other data including music data received via, e.g., the global Internet. The software module could reside in RAM memory, flash memory, registers, or any other form of writable storage medium known in the art. The memory device 1151 may be, but not limited to, a single memory, CD, DVD, ROM, RAM, EEPROM, optical storage, magnetic disk storage, flash memory storage, or any other non-volatile storage medium capable of storing digital data.

An optionally incorporated SIM card 1149 carries, for instance, important information, such as the cellular phone number, the carrier supplying service, subscription details, and security information. The SIM card 1149 serves primarily to identify the mobile terminal 1101 on a radio network. The card 1149 also contains a memory for storing a personal telephone number registry, text messages, and user specific mobile terminal settings.

In some embodiments, the mobile terminal 1101 includes a digital camera comprising an array of optical detectors, such as charge coupled device (CCD) array 1165. The output of the array is image data that is transferred to the MCU for further processing or storage in the memory 1151 or both. In the illustrated embodiment, the light impinges on the optical array through a lens 1163, such as a pin-hole lens or a material lens made of an optical grade glass or plastic material. In the illustrated embodiment, the mobile terminal 1101 includes a light source 1161, such as a LED to illuminate a subject for capture by the optical array, e.g., CCD 1165. The light source is powered by the battery interface and power control module 1120 and controlled by the MCU 1103 based on instructions stored or loaded into the MCU 1103.

### 4. ALTERNATIVES, DEVIATIONS AND MODIFICATIONS

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

### 5. REFERENCES

1. World Health Organization. WHO Coronavirus (COVID-2019) dashboard. https://covid19.who.int/(2021).
2. van Doremalen, N. et al. Aerosol and surface stability of SARS-CoV-2 as compared with SARS-CoV-1. N. Engl. J. Med. 382, 1564-1567 (2020).
3. Centers for Disease Control and Prevention. Community use of cloth masks to control the spread of SARS-CoV-2. Science Briefs https://www.cdc.gov/coronavirus/2019-ncov/science/science-briefs/masking-science-sars-cov2.html (2020).
4. Lindsley, W. G., Blachere, F. M., Law, B. F., Beezhold, D. H., Noti, J. D. Efficacy of face masks, neck gaiters and face shields for reducing the expulsion of simulated cough-generated aerosols. Aerosol Sci. and Tech. 55, 449-457 (2021).
5. Centers for Disease Control and Prevention. How to wear masks. https://www.cdc.gov/coronavirus/2019-ncov/prevent-getting-sick/how-to-wear-cloth-face-coverings.html (2021).
6. United Nations News. Five things you should know about disposable masks and plastic pollution. Climate and Environment https://news.un.org/en/story/2020/07/1069151 (2020).
7. Sangkham, S. Face mask and medical waste disposal during the novel COVID-19 pandemic in Asia. Case Studies in Chemical and Environmental Eng. 2, 100052; 10.1016%2Fj.cscee.2020.100052 (2020).
8. Ilyas, S., Srivastava, R., Kim, H. Disinfection technology and strategies for COVID-19 hospital and bio-medical waste management. Sci. Total Environ. 749, 141652; 10.1016%2Fj.scitotenv.2020.141652 (2020).
9. World Health Organization. Health care waste. WHO Fact Sheets https://www.who.int/news-room/fact-sheets/detail/health-care-waste (2018).
10. World Health Organization. Shortage of personal protective equipment endangering healthcare workers worldwide. WHO News https://www.who.int/news/item/03-03-2020-shortage-of-personal-protective-equipment-endangering-health-workers-worldwide (2020).
11. Williams, C. M. Exhaled Mycobacterium tuberculosis output and detection of subclinical disease by face-mask sampling: prospective observational studies. The Lancet Infectious Diseases. 20, 607-617 (2020).
12. Centers for Disease Control and Prevention. Cleaning and disinfecting your facility. Cleaning, Disinfecting, and Ventilation https://www.cdc.gov/coronavirus/2019-ncov/community/disinfecting-building-facility.html (2021).
13. Meechan, P. J., Potts, J. Laboratory safety level criteria in Biosafety in microbiological and biomedical laboratories, 6^{th} ed. 32-69 (U.S. Department of Health and Human Services, 2020).
14. United States Food and Drug Administration. UV lights and lamps: Ultraviolet-C radiation, disinfection, and Coronavirus. Coronavirus (COVID-19) and Medical Devices https://www.fda.gov/medical-devices/coronavirus-covid-19-and-medical-devices/uv-lights-and-lamps-ultraviolet-c-radiation-disinfection-and-coronavirus (2021).
15. Storm, N. et. al. Rapid and complete inactivation of SARS-CoV-2 by ultraviolet-C irradiation. Sci. Rep. 10, 22421; 10.1038/s41598-020-79600-8 (2020).
16. Buonanno, M., Welch, D., Shuryak, I., Brenner, D. J. Far-UVC light (222 nm) efficiently and safely inactivates airborne human coronaviruses. Sci. Rep. 10, 10285; 10.1038/s41598-020-67211-2 (2020).
17. Collins, F. M. Relative susceptibility of acid-fast and non-acid-fast bacteria to ultraviolet light. Appl. Microbiol. 21, 411-413 (1971).
18. Brais, N. Effect of germicidal UV on plastic materials. Sanuvox blog https://sanuvox.com/blog/effect-of-germicidal-uv-on-plastic-materials/(2017).
19. Waveform Lighting. cleanUV™ 270 nm LED strip light irradiance pattern. https://www.waveformlighting.com/datasheets/BP7026.pdf (2021).
20. Pathak, P., Park, S., & Cho, H. J. A carbon nanotube-metal oxide hybrid material for visible-blind flexible UV-sensor. Micromachines. 11, 368; 10.3390/mi11040368 (2020).

What is claimed is:

1. An apparatus comprising:
a housing defining an interior space configured to receive a mask sheet such that the mask sheet is movable from a first position covering a portion of a face of a user to a second position within the interior space of the housing; and
a light source mounted within the interior space and configured to emit radiation to sterilize the mask sheet in the second position.

2. The apparatus of claim 1, wherein the housing is configured to receive the mask sheet such that the mask sheet takes an arcuate path within the interior space of the housing as the mask sheet is moved from the first position to the second position within the interior space.

3. The apparatus of claim 2, wherein the housing is configured to receive the mask sheet such that the mask sheet takes a serpentine path comprising a plurality of turns within the interior space of the housing as the mask sheet is moved from the first position to the second position within the interior space.

4. The apparatus of claim 2, further comprising a pair of spaced apart layers positioned within the interior space that define the arcuate path between the pair of spaced apart layers.

5. The apparatus of claim 4, wherein the spaced apart layers are made from material that is transmissible of the radiation emitted by the light source.

6. The apparatus of claim 4, wherein the pair of spaced apart layers each have a serpentine shape such that the arcuate path between the pair of spaced apart layers is a serpentine path comprising a plurality of turns.

7. The apparatus of claim 2, further comprising one or more rods mounted within the interior space such that the mask sheet is configured to take the arcuate path as the mask sheet moves over the one or more rods.

8. The apparatus of claim 7, wherein the one or more rods are made from material that is transmissible of the radiation emitted by the light source.

9. The apparatus of claim 7, wherein the one or more rods are a plurality of rods mounted within the interior space such that the mask sheet is configured to take a serpentine path comprising a plurality of turns based on the mask sheet moving over the plurality of rods.

10. The apparatus of claim 2, wherein an inner rod is pivotally mounted within the interior space and the mask sheet is configured to wrap around an outer surface of the inner rod as the mask sheet is moved from the first position to the second position.

11. The apparatus of claim 2, wherein the light source includes a plurality of light sources mounted within the interior space.

12. The apparatus of claim 1, further comprising:
an inner rod rotatably mounted within the interior space;
a spring-loaded ratchet gear to rotatably mount the inner rod within the housing; and
a pawl configured to engage the ratchet gear when the mask is in the first position.

13. The apparatus of claim 12, further comprising a switch on the housing and operatively coupled to the pawl such that engagement of the switch is configured to disengage the pawl from the ratchet gear to move the mask sheet from the first position to the second position.

14. The apparatus of claim 1, wherein the light source is an ultraviolet (UV) light source.

15. The apparatus of claim 14, wherein the UV light source is a UV light emitting diode (LED) panel arranged within the interior space such that UV light emitted from the UV LED panel is directed at the mask sheet.

16. The apparatus of claim 1, further comprising:
a controller communicatively coupled with the light source;
a sensor configured to detect that the mask sheet has moved from the first position to the second position, wherein the sensor is communicatively coupled with the controller and is configured to transmit a first signal to the controller upon detection of the mask sheet moving to the second position;
wherein upon receiving the first signal from the sensor the controller is configured to transmit a second signal to the light source to cause the light source to emit radiation to sterilize the mask sheet in the second position.

17. The apparatus of claim 1, wherein the housing defines a slot through which the mask sheet is configured to pass through in moving from the first position to the second position and wherein the apparatus further includes a cover that is configured to move from a first position not covering the slot when the mask sheet is in the first position to a second position covering the slot when the mask sheet is in the second position.

18. A cartridge to replace one or more components of the apparatus of claim 1, said replaceable cartridge comprising the mask sheet.

19. A method comprising:
positioning the apparatus of claim 1 on the user;
doffing the mask sheet including moving the mask sheet from the first position covering the portion of the face of the user to the second position within the interior space of the housing; and
emitting radiation from the light source within the interior space to sterilize the mask sheet in the second position.

20. The method of claim 19, further comprising donning the mask sheet including moving the mask sheet from the second position within the housing to the first position such that the mask sheet covers the portion of the face of the user.
